(19) United States
(12) Patent Application Publication
(10) Pub. No.: US 2026/0308518 A1
(43) Pub. Date: Oct. 8, 2026

(54) METABOLICALLY ENHANCED VD1 T CELLS AND USES THEREOF

(71) Applicant: The Brigham and Women's Hospital, Inc., Boston, MA (US)

(72) Inventors: Lydia Lynch, Newton Highlands, MA (US); Cathal Harmon, Allston, MA (US)

(21) Appl. No.: 19/484,161

(22) PCT Filed: May 16, 2024
(86) PCT No.: PCT/US2024/029728

§ 371 Date: Nov. 13, 2025

Related U.S. Application Data
(60) Provisional application No. 63/466,826, filed on May 16, 2023.

(51) Int. Cl. C12N 5/0783; A61K 35/17; A61P 35/00; G01N 33/569

(52) U.S. Cl. CPC C12N 5/0636; A61K 35/17; A61P 35/00; G01N 33/56972; C12N 2500/32; C12N 2501/2301; C12N 2501/2304; C12N 2501/2315; C12N 2501/2321; C12N 2501/24; C12N 2501/515; C12N 2501/53

(57) ABSTRACT

Described herein are Vδ1 T cells that are cytotoxic in nature and exhibit improved metabolic activity. Also described here are methods for expansion of such Vδ1 T cells. Further provided are use of such Vδ1 T cells in methods of treatment.

### CLAIM OF PRIORITY

This application claims the benefit of U.S. Provisional Application Ser. No. 63/466,826, filed on May 16, 2023. The entire contents of the foregoing are incorporated herein by reference.

### TECHNICAL FIELD

The present disclosure relates to cytotoxic gamma delta (γδ) T cells and methods for expansion of such cells.

### BACKGROUND

Mammalian tissues are populated by a range of resident innate lymphocytes including gd T cells, MAIT cells, innate lymphoid cell (ILCs), as well as myeloid cells. In addition to classical immunosurveillance, these cells also perform homeostatic duties, such as regulating tissue architecture and turnover of epithelial and stromal cells (see, e.g., Nielsen et al., Nat. Rev Immunol. 17, 733-745 (2017); Melsen et al., Front. Immunol. 7, 1-10 (2016); Schenkel et al., Immunity 41, 886-897 (2014); Vivier et al., Cell 174, 1054-1066 (2018); Suzuki et al., Immunol. Rev 298, 198-217 (2020)). How these innate tissue resident T cells change in situ as tumors develop is unknown. Gamma delta (γδ) T cells are innate lymphocytes which recognize a range of non-classical MHC molecules and stress induced ligands, and are poised to rapidly respond to changes in homeostasis (Raverdeau et al., Clin. Transl. Immunol. 8, (2019)). Of the innate T cell subsets, γδ T cells have been most frequently associated with improved prognosis in solid tumors (Gentles et al., Nat. Med. 21, 938-945 (2015)). γδ T cells have several important features that make them ideal candidates for adoptive cell therapy. γδ T cells can kill tumors via degranulation of cytotoxic factors, and secrete proinflammatory cytokines and transactivate other anti-tumor cells. Unlike CD8 T cells, γδ T cells can be activated independent of their TCR, eliminating the need for identifying tumor specific antigens in advance. However, γδ T cells are heterogeneous and little is known about their heterogeneity in humans and whether all 16 T cells are beneficial against cancer.

In mice, two developmentally and functionally distinct subsets of γδ T cells exist, those that produce IFNγ and those that produce IL-17. However, in humans these distinctions are less clear, and the production of IL-17 by human γδ T cells remains controversial. Although some evidence for IL-17 production in colon cancer has previously been shown, high variability in the number of these cells was noted (Wu et al., Cell. Mol. Immunol. 14, 245-253 (2017); Wu et al., Immunity 40, 785-800 (2014)). Human γδ T cells are broadly divided into three main subtypes based on delta chain usage Vδ1, Vδ2 and Vδ3. However, functionally they appear similar in blood, with all subsets capable of cytotoxic function and IFNγ production (Holderness et al., Annu. Rev. Anim. Biosci. 1, 99-124 (2013); Raverdeau et al., Clin. Transl. Immunol. 8, 1-15 (2019)). Much of the current literature focuses on Vδ2 cells, due to their ease of access in the blood. Some Vδ2 recognize phosphoantigens, and have so far shown limited success in an immunotherapy setting (Hoeres et al., Front. Immunol. 9, 1-18 (2018)). Vδ1 and Vδ3 cells are more tissue resident and so may act early in cancer at the site of development. In a pan-cancer assessment of the TCGA database, γδ T cells were identified as the strongest positive prognostic across all solid cancer in 18,000 patients (Thorsson et al., Immunity 48, 812-830.e14 (2018); Gentles et al., Nat. Med. 21, 938-945 (2015)). A limitation of this study was the ability of the CIBERSORT algorithm to differentiate γδ T cells from CD8 T cells. This was further refined by the lab of Jean Jacques Fournie (Meraviglia et al., Oncoimmunology 6, (2017)), with similar results showing the prognostic value of γδ T cells. However, these studies did not discriminate between γδ T cell subsets, and CRC was identified as a site where γδ T cells were not associated with improved prognosis. Therefore, there is an unmet need to know if there is a difference in γδ T cell subsets in CRC compared to other tumor type where they were associated with positive prognosis, namely endometrial cancer.

### SUMMARY

The present disclosure provides gamma delta (γδ) T cells, in particular, γδ T cells of Vδ1 subtype (Vδ1 T cells), that are cytotoxic in nature, and exhibit metabolic flexibility and persistence in vivo. Also described herein are methods for expansion of Vδ1 T cells and methods of treatment (e.g., methods of treating cancer) using such cells.

Accordingly, in a first aspect, the present disclosure provides a method for expansion of a population of Vδ1 T cells by: (i) providing a population of cells comprising Vδ1 T cells isolated from a human subject; (ii) culturing the Vδ1 T cells from step (i) in a first medium comprising human serum, interleukin 1β (IL-1β), IL-4, IL-21, interferon gamma (IFNγ), and an anti-CD3 antibody or an antigen-binding fragment thereof, for 10-20 days, optionally wherein the first medium further comprises glutamine; and (iii) culturing the cells in a second medium comprising human serum, IL-15, IFNγ, IL18, an anti-CD2 antibody or an antigen-binding fragment thereof, and an anti-CD3 antibody or an antigen-binding fragment thereof, for 3-10 days, optionally wherein the second medium further comprises glutamine.

In some embodiments, the population of Vδ1 T cells are isolated from a biological sample from the human subject. In certain embodiments, isolating the population of Vδ1 T cells from the biological sample comprises isolation of a population of peripheral blood mononuclear cells (PBMCs) from the biological sample and depletion of monocytes, macrophages, and/or alpha beta T cells from the population of PBMCs. In some instances, isolation of the population of PBMCs from the biological sample comprises density gradient centrifugation. In certain embodiments, the biological sample comprises blood.

In some embodiments, the method further comprising identification of the Vδ1 T cells by immunophenotyping. In certain embodiments, the identification of the Vδ1 T cells comprises detecting the presence of one or more of TCR Vδ1, CD3, NKG2D, γδTCR, and TIGIT on the Vδ1 T cells. In certain embodiments, the identification of the Vδ1 T cells comprises detecting the absence of one or more of CD14, CD68, CD19, CD21, CD56, and CD4 on the Vδ1 T cells. In some instances, the immunophenotyping comprises flow cytometry or immunofluorescence microscopy.

In some embodiments, the cells are cultured in the first medium for 14 or 15 days.

In some embodiments, the cells are cultured in the second medium for 7 or 8 days.

In some embodiments, the first medium comprises about 1-10% human serum, about 10-20 ng/ml IL-1β, about 50-150 ng/ml IL-4, about 1-15 ng/ml IL-21, about 10-150 ng/ml IFNγ, and about 10-150 ng/ml of the anti-CD3 antibody or antigen-binding fragment thereof. In certain embodiments, the first medium comprises about 5% human serum about 15 ng/ml IL-1β, about 100 ng/ml IL-4, about 7 ng/ml IL-21, about 70 ng/ml IFNγ, and about 70 ng/ml of the anti-CD3 antibody or antigen-binding fragment thereof. In some embodiments, the first medium further comprises glutamine. In some embodiments, the first medium comprises about 1-5 mmol glutamine. In certain embodiments, the first medium comprises about 2 mmol glutamine. In some embodiments, the first medium does not comprise glutamine.

In some embodiments, the second medium comprises about 0.1%-5% human serum, about 10-150 ng/ml IL-15, about 1-50 ng/ml IFNγ, about 1-100 ng/ml IL-18, about 100-1000 ng/ml of the anti-CD2 antibody or antigen-binding fragment thereof, and about 0.1-10 μg/ml of the anti-CD3 antibody or antigen-binding fragment thereof. In certain embodiments, the second medium comprises about 1% human serum, about 70 ng/ml IL-15, about 30 ng/ml IFNγ, about 50 ng/ml IL-18, about 500 ng/ml of the anti-CD2 antibody or antigen-binding fragment thereof, and about 1 μg/ml of the anti-CD3 antibody or antigen-binding fragment thereof. In some embodiments, the second medium further comprises glutamine. In some embodiments, the second medium comprises about 1-5 mmol glutamine. In certain embodiments, the second medium comprises about 2 mmol glutamine. In some embodiments, the second medium does not comprise glutamine.

In another aspect, the present disclosure provides an expanded population of Vδ1 T cells obtained by the method described hereinabove.

In some embodiments, 35% or more cells of the population express T cell immunoreceptors with Ig and ITIM domains (TIGIT). In some embodiments, 35% or more cells of the population express IFNγ. In some embodiments, 20% or less cells of the population express NKp80. In some embodiments, 20% or less cells of the population express AREG.

In some embodiments, 35% or more cells of the population express cytotoxic effector molecules when co-incubated with cancer cells. In certain embodiments, the cytotoxic effector molecules comprise one or more of granzyme B, perforin, IFNγ, CD107a, TNFα, RANTES, granzyme K, TRAIL, FAS-FASL, and granulysin.

In some embodiments, the cells of the population do not show an increase in mitochondrial ROS production under hypoxic and/or acidic conditions, as compared to mitochondrial ROS production by the cells under normal conditions.

In some embodiments, the cells of the population do not show a decrease in cytotoxicity under hypoxic and/or acidic conditions, as compared to cytotoxicity of the cells under normal conditions.

In another aspect, the present disclosure provides a method of treating cancer in a subject in need thereof by administering to the subject a therapeutically effective amount of the expanded population of Vδ1 T cells described hereinabove.

In some embodiments, the population of Vδ1 T cells is a population of allogeneic Vδ1 T cells. In some embodiments, the population of Vδ1 T cells is a population of autologous Vδ1 T cells.

In some embodiments, the cancer is a solid tumor.

In some embodiments, the subject is a human.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

FIGS. 1A-1I: Differences in prognostic value of Vd1 T cells is not due to exhaustion. FIGS. 1A-1D show survival analysis of ovarian (OV; FIG. 1A), lung (LUAD; FIG. 1B), endometrial (UCEC; FIG. 1C) and colorectal (COAD; FIG. 1D) cancer patients from the TCGA database, stratified by high and low expression of γδ TCR genes TRDV1 & TRDV2. FIG. 1E provides representative FACS plots of Vδ1 T cells in healthy endometrium and endometrial tumors. FIG. 1F shows percentage of Vδ1 & Vδ2 T cells in healthy endometrium and endometrial tumors. FIG. 1G provides representative FACS plots of Vd1 T cells in healthy colon and colorectal tumors. FIG. 1H shows percentage of Vd1 & Vd2 T cells in healthy colon and colorectal tumors. FIG. 1I provides uMAP representation of single cell RNAseq data from colorectal and endometrial tumors. Data presented as mean±SEM. Data were analyzed using Cox Proportional Hazards Model (A-D), Wilcoxon matched pairs test (F&H) (A-D n=379-575, F n=9-15, H n=7, I CRC n=7, Endo n=2, Flagship CRC n=80, *p<0.05, **, p<0.01).

FIGS. 2A-2K: Tumor infiltrating gd T cells are not exhausted and maintain cytotoxic phenotype. FIG. 2A is a heatmap showing gene expression of immune checkpoints in cell subsets in colorectal and endometrial tumors. FIG. 2B provides representative FACS plots of PD-1 expression in CD8, Vd1, Vd2 and NK cells from colorectal tumors. FIG. 2C shows percentage of CD8, Vd1, Vd2 and NK cells positive for PD-1. FIG. 2D provides representative FACS plots of TIGIT expression in CD8, Vd1, Vd2 and NK cells from colorectal tumors. FIG. 2E shows percentage of CD8, Vd1, Vd2 and NK cells positive for TIGIT. FIGS. 2F-2G show percentage of cell subsets expressing immune checkpoints LAG3 & TIM3. FIGS. 2H-2K show percentage of cell subsets expressing effector molecules GZMB, GZMK, IFNg & TNFa. Data presented as mean±SEM. Data were analyzed using Friedman test, with Dunn's multiple comparison test. (F-K n=11, *p<0.05).

FIGS. 3A-3I: γδ T cells in colorectal cancer are heterogeneous compared to endometrial cancer. FIG. 3A provides uMAP plot of gd T cells from endometrial and colorectal cancer, showing differences in cell clusters between tumor types. FIG. 3B provides a heatmap showing the top 15 differentially expressed genes between gd T cell clusters in colorectal cancer. FIG. 3C provides Z-score of expression of immune checkpoints calculated and plotted for gd T cell clusters from CRC. FIG. 3D provides gene expression of AREG, IFNG in gd subsets from scRNAseq of colorectal tumors. FIG. 3E provides gene expression of AREG, IFNG, GZMB & PRF1 in gd T cells subsets from scRNAseq analysis of colorectal cancer. FIG. 3F provides representative FACS plots of IFNg and AREG expression in Vd1, Vd2 & Vd3 T cells. FIGS. 3G-3I provides percentage of AREG+(FIG. 3G), IFNg+(FIG. 3H) and AREG+IFNg+(FIG. 3I) Vd1, Vd2 & Vd3 T cells. Data presented as mean±SEM. Data were analyzed using Friedman test, with Dunn's multiple comparison test (G n=15, *p<0.05, **, p<0.01, ***p<0.001, ****, p<0.0001).

FIGS. 4A-4O: AREG is associated with tumors characterized by wound healing, present in healthy colon, and upregulated in response to malignancy in gd T cells alone. FIG. 4A-4B provide gene expression data for AREG in normal colon, CRC tumors, normal endometrium, and endometrial tumors from TCGA and GTEx datasets. FIG. 4C provides gene expression data for AREG in CRC tumors stratified by microsatellite status (MSS-microsatellite stable, MSI-microsatellite instability, H-high. L-low). FIG. 4D provides gene expression data for AREG in CRC tumors stratified by consensus molecular classification for colon cancer. FIGS. 4E-4H provide uMAP plot of lymphocyte subsets in healthy colon & CRC tumor, stratified by sample type, showing AREG & IFNG expression. FIG. 4I shows mean expression of AREG in immune cell clusters in healthy colon (“H”) and CRC tumor (“C”) from the Flagship CRC dataset. FIG. 4J shows proportion of immune subsets expressing AREG in healthy colon (“H”) and CRC tumor (“C”). FIG. 4K shows proportion of immune subsets expressing IFNG in healthy colon (“H”) and CRC tumor (“C”). FIG. 4L provides uMAP plot of gd T cell subsets in normal colon tissue and CRC tumor stratified by mismatch repair status (MMRp—mismatch repair proficient, MMRd—mismatch repair deficient). FIG. 4M shows proportions of gd T cell subsets in normal colon, MMRp & MMRd colon tumors. FIGS. 4N-4O show gene expression of AREG & IFNG in gd T cell subsets in normal colon, MMRp & MMRd CRC tumors. Data presented as mean±SEM. Data were analyzed using Mann Whitney U test (A-B) Friedman test, with Dunn's multiple comparison test (C-D, I-K) (K n=15, *p<0.05, **, p<0.01, ****, p<0.000l).

FIGS. 5A-5L: AREG is associated with NKp80+ γδ T cells and induces proliferation of CRC tumor cells. FIGS. 5A-5B show gene expression of KLRF1 & TIGIT in γδ T cell subsets in CRC tumors. FIG. 5C shows percentage expression of NKp80 & TIGIT in Vδ1, Vδ2 & Vδ3 T cells in CRC tumors. FIG. 5D provides representative FACS plot of AREG & IFNγ expression in Vδ1 T cells subsetted by NKp80 & TIGIT expression after 4 hr PMA stimulation. FIG. 5E shows percentage of IFNγ & AREG expression in Vδ1 T cells subsetted by NKp80 & TIGIT expression. FIG. 5F shows percentage of Vδ1 T cells expressing AREG pre- and post-expansion. FIG. 5G shows percentage of Vδ1 T cells expressing NKp80 pre- and post-expansion. FIG. 5H provides representative images of scratch assay using SW480 cell line at 0 & 24 hours±co-incubation with Vδ1 cells. FIG. 5I shows percentage of scratch area at 0, 6 & 24 hrs, untreated or treated with Vδ1 cells. FIG. 5J shows percentage of Vδ1 T cells expressing AREG after 24 hours co-incubation with scratched and unscratched SW480 cells. FIG. 5K shows concentration of AREG in supernatants of SW480 cells without Vδ1 treatment (Control) or with Vδ1 T cells with or without cell scratching. FIG. 5L shows percentage of scratch area of SW480 cells at 24 hrs untreated, treated with Vδ1 T cells cetuximab (αEGFR), or aTCRγδ. Data presented as mean±SEM. Data were analyzed using Friedman test, with Dunn's multiple comparison test (C, E, J, M), Wilcoxon matched pairs test (G-H, K) (C, E n=8-15, F-G n=5, I n=8, J n=5, K n=3, L n=4, *p<0.05, **p<0.01, ***p<0.001, ****p<0.0001).

FIGS. 6A-6R: AREG production is enhanced by pro-inflammatory cytokines and can be inhibited in Gen 2 cells. FIG. 6A: Fold change in cell numbers after expansion using Gen 1 or Gen 2 protocols. FIG. 6B: Expression of NK receptors: CD16, NKG2D, NKp44, NKp46 & NKp80 in Vd1 T cells expanded using Gen 1 or Gen 2 protocols. FIG. 6C: Representative FACS plots of memory phenotype (Naïve—CD27A+CD45RA+, Central Memory—CD27+CD45RA−, Effector Memory—CD27−CD45RA−, Terminally Differentiated Effector Memory—CD27−CD45RA+). FIG. 6D: Memory phenotype of Vd1 T cells expanded using Gen 1 or Gen 2 protocols. FIGS. 6E-6H: Expression of immune checkpoints TIGIT, TIM3, LAG3 & PD-1 in Vd1 T cells expanded using Gen 1 or Gen 2 protocols. FIG. 6I: Vd1 T cells expanded using Gen 1 or Gen 2 protocols were co-incubated with K562 tumor cells for 4 hours, and direct cytotoxicity was assessed. Percentage of target cells killed by Gen 1 or Gen 2 cells is shown. FIGS. 6J-6K: Gen 1 or Gen 2 cells were co-incubated with SW480 tumor cells for 4 hours, and direct cytotoxicity was assessed. FIG. 6J shows percentage of target cells killed by Gen 1 or Gen 2 cells; FIG. 6K shows percentage of Gen 1 or Gen 2 cells expressing CD107a. FIG. 6L: Representative FACS plots of AREG & IFNγ in Gen 1 or Gen 2 cells after 4-hour PMA stimulation. FIGS. 6M-6N: Percentage of IFNγ & AREG positive Gen 1 or Gen 2 cells. FIGS. 6O-6R: Representative images of scratch assay using SW480 cell line at 0 & 24 hours±co-incubation with Gen 1 or Gen 2 cells. FIG. 6O shows percentage of scratch area of SW480 cells imaged after 6 or 24 hours co-incubation with Gen 1 or Gen 2 cells. FIG. 6P shows percentage of Vδ1 T cells expressing degranulation marker CD107a after 24-hour co-incubation with SW480 cells. FIG. 6Q shows percentage of Vδ1 T cells producing IFNγ after 24-hour co-incubation with SW480 cells. FIG. 6R shows percentage of Vδ1 T cells producing AREG after 24-hour co-incubation with SW480 cells. Data presented as mean±SEM. Data were analyzed using Friedman test, with Dunn's multiple comparison test (B, D, J-K, P, Q, R), Wilcoxon matched pairs test (A, C, E-H, I, M-N) (A n=23, B n=7-9, D n=14, E-H n=6-10, I n=10, J-K n=5, M-N n=13, P n=8, Q-R n=5, *p<0.05, **, p<0.01, ***, p<0.001).

FIGS. 7A-7S: Characterization of metabolic advantages of Gen 2 cells. FIG. 7A: Representative FACS plot of Mitotracker Green (MTG) and Tetramethylrhodamine methyl ester (TMRM) in Vd1 T cells expanded using Gen 1 & Gen 2 protocols. FIG. 7B: Mean Fluorescence intensity (MFI) of MTG in Gen 1 & Gen 2 cells. FIG. 7C: MFI of TMRM in Gen 1 & Gen 2 cells. FIG. 7D: Extracellular acidification rate (ECAR) of Gen 1 & Gen 2 cells basally and in response to metabolic inhibitors indicated. FIG. 7E: Histogram of basal glycolysis in Gen 1 & Gen 2 cells. FIG. 7F: Histogram of glycolytic capacity of Gen 1 & Gen 2 cells. FIG. 7G: Oxygen consumption rate (OCR) of Gen 1 & Gen 2 cells basally and in response to metabolic inhibitors indicated. FIG. 7H: Basal oxidative phosphorylation rate in Gen 1 & Gen 2 cells. FIG. 7I: Maximum respiratory rate in Gen 1 & Gen 2 cells. FIGS. 7J-7M: Expression of metabolic transporters GLUT1, CD36, CD39 and CD71 in Gen 1 & Gen 2 cells. FIGS. 7N-7Q: Expression of metabolic genesMYC, SLC27A3, SLC7a5 & SLCa5 in Gen 1 & Gen 2 cells. FIGS. 7R-7S: Gen 1 & Gen 2 cells were incubated for 24 hours with either lactic acid (10-20 mM) or in hypoxic conditions (0.5% O2) and activated for 4 hours with PMA. FIG. 7R shows percentage of Gen 1 & Gen 2 cells expressing mitochondrial ROS (mitosox) after treatment. FIG. 7S shows percentage of Gen 1 & Gen 2 cells expressing IFNγ after treatment. Data presented as mean±SEM. Data were analyzed using Wilcoxon matched pairs test (B-C, E-F, H-P) or using Friedman test, with Dunn's multiple comparison test (Q-S) (B-C n=11, D-P n=5, Q-S n=4-5, *p<0.05, **p<0.01).

FIGS. 8A-8L: GEN 2 cells inhibit PDX tumor growth and do not produce AREG in the tumor microenvironment. FIG. 8A: Schematic of humanized tumor model. NSG-Tg(HuIL15) were injected with patient derived xenografts (PDX) of human CRC tumor cells and allowed to grow for 6 weeks Expanded Vd1 T cells (10×10⁶/mouse) were injected intravenously. After 7 days, tumor growth was measured and cells isolated from tumors, spleen, lung, bone marrow (BM) & blood. FIG. 8B: Tumor growth curves for mice treated with PBS, Gen 1 cells or GEN 2 cells. Cells were injected at 49 days post tumor implantation. FIG. 8C: Tumor volume at day 56 post implantation. FIG. 8D: Representative FACS plots of Vd1 T cell infiltration of PDX tumors by Gen 1 and Gen 2 cells. FIG. 8E: Quantification of absolute cell numbers of tumor infiltrating Gen 1 and GEN 2 cells. FIG. 8F: Quantification of absolute cell counts of Gen 1 and GEN 2 cells in peripheral organs: spleen, lungs, bone marrow (BM) & blood. FIG. 8G: Expression of nutrient transporters CD39, CD71, CD98 & GLUT1 in tumor infiltrating Gen 1 & Gen 2 cells. FIG. 8H: MFI of MTG & TMRM in tumor infiltrating Gen 1 & Gen 2 cells. FIG. 8I: Expression of AREG positive Gen 1 & Gen 2 cells in peripheral organs (blood, spleen, bone marrow, lung) and PDX tumors. FIG. 8J: Representative FACS plots of AREG and IFNg production in tumor infiltrating Gen 1 & Gen 2 cells. FIG. 8K: Percentage of Gen 1 & Gen 2 cells expressing IFNg. FIG. 8L: Percentage of Gen 1 & Gen 2 cells expressing AREG. Data presented as mean±SEM. Data were analyzed using Friedman test, with Dunn's multiple comparison test (C, F, G, J), Wilcoxon matched pairs test (E, I, L) (B-F n=7, G-L n=4, *p<0.05, **, p<0.01, ***, p<0.001).

FIG. 9: AREG (left panel) and IFNg (right panel) production in Vδ1 T cells that were expanded using T cell media, human plasma-like media (HPLM) alone, HPLM supplemented with excess glucose (HPLM+Glu), HPLM supplemented with glutamine (HPLM+Glut), or HPLM supplemented with non-essential MEM amino acids (HPLM+AA) (*p<0.05, **, p<0.01).

### DETAILED DESCRIPTION

Described herein are gamma delta (γδ) T cells, in particular, γδ T cells of Vδ1 subtype (Vδ1 T cells), that are cytotoxic in nature, and exhibit metabolic flexibility and persistence in vivo. Also described herein are methods for expansion of such Vδ1 T cells. Further provided are methods of treatment (e.g., methods of treating cancer) using such cells.

Immune cells represent an important target for therapies in malignancy. However, only a small proportion of patients with solid tumors respond to these therapies. A possible reason for this could be the fact that focus of most of these therapies is adaptive immune response, such as those involving the CD8+ T cells. The present disclosure determines that genes associated with the innate immune response—specifically, those associated with gamma delta (γδ) T cells, and in particular, γδ T cells of Vδ1 subtype (Vδ1 T cells)—display the strongest prognostic value. Vδ1 T cells are an attractive cell type for adoptive therapy, as they are not MHC restricted and therefore graft-versus-host disease (GvHD) is not likely to occur. In addition, they naturally home to mucosal surfaces, such as the gut and endometrium, so would therefore more effectively traffic to the site of tumor growth. Another important feature of Vδ1 T cells for therapy is their natural ability to home to tissue, making them ideal to target solid tumors. However, the literature is sparse regarding the role of Vδ1 T cells in solid cancers.

The present disclosure describes an enrichment of Vδ1 T cells in solid tumors, including colon and endometrial cancers. The present disclosure also identifies that Vδ1 T cells can be divided into two functional subsets in the tumor—an anti-tumor (IFNγ producing) subset, and a pro-tumor (amphiregulin (AREG) producing) subset. Both of these subsets are tissue resident, and can be found in the healthy gut and endometrial mucosa. Moreover, the present disclosure describes surface markers that can be used to identify these subsets: TIGIT marking the anti-tumor subset, and NKp80 marking the pro-tumor subset. Unlike conventional immune checkpoints, TIGIT appears to mark cells that have been ‘licensed’ and are significantly more cytotoxic than ‘unlicensed’ naive cells. Also described here is a protocol to expand Vδ1 T cells, in particular, enriching for TIGIT positive Vδ1 T cells and eliminating AREG producing Vδ1 T cells. The expanded population of Vδ1 T cells therefore display superior cytotoxicity and do not produce factors that might aid tumor growth. This protocol differs significantly from currently published literature. In addition, described here is a method for metabolically reprogramming these cells, such that the cells are able to survive longer in vivo, which has been demonstrated in humanized mouse models. This is due to enhanced mitochondrial dynamics. Using patient-derived xenografts in humanized mice, the present disclosure determines that these cells show enhanced tumor control, longevity and metabolic activity compared to cells expanded using protocols known in the art. As described herein, these cells are applicable across a number of malignancies and can be used, e.g., as an off the shelf first line immunotherapy. The present methods achieved a 600 fold expansion (increased significantly from 20 fold) and provide a protocol for further expansion without compromising quality.

As demonstrated in the Examples hereinafter, using extensive scRNAseq data and flow cytometry, two functional subsets of γδ T cells were identified in human mucosal tumors, one with an AREG producing, wound healing phenotype; and the other with high cytotoxicity. The presence of these two opposing γδ T cell subsets depends on the tumor type. Only the cytotoxic subset is present in endometrial cancer, while colorectal cancer (CRC) contains both subsets. The AREG producing subset showed wound healing, pro-tumor functions toward human CRC cells. However, despite this, Vδ1 still hold immense potential for adoptive cell therapy. The other functional population of Vδ1 in tumors have high cytotoxic features with low checkpoint expression. The present disclosure provides the first complete functional and metabolic characterization of γδ T cells in solid tumors, and a protocol for enhancing the cytotoxic subset while suppressing the AREG subset. Using the methods described here, superior expanded Vδ1 cells can be obtained that have enhanced anti-tumor function, cytotoxicity, metabolic flexibility, and persistence in vivo. This enhanced product demonstrated improved infiltration/survival in tumors and peripheral tissues in humanized models of CRC and could slow tumor growth in established CRC tumors in vivo, as demonstrated in the Examples section.

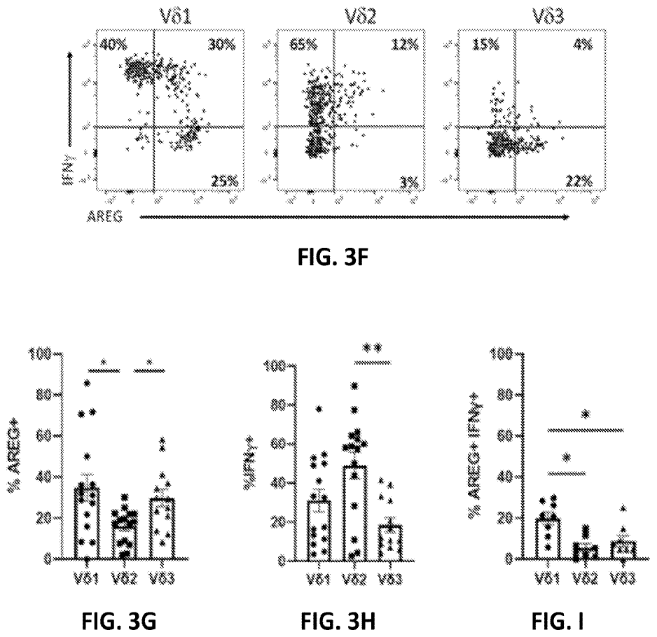

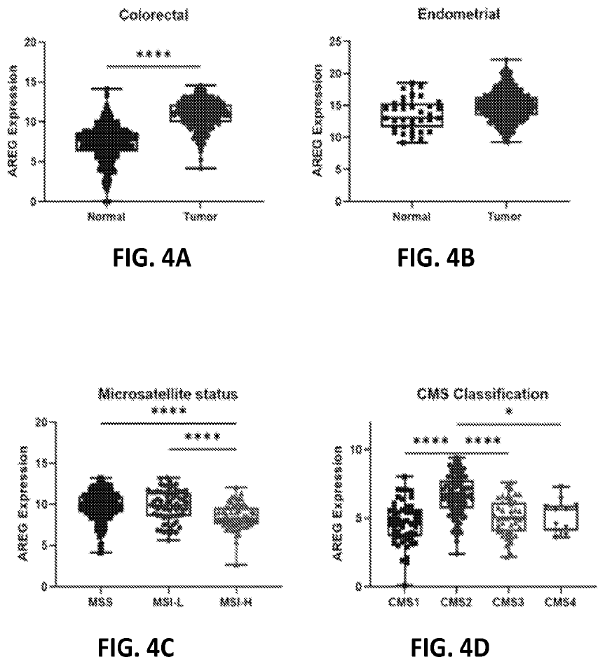

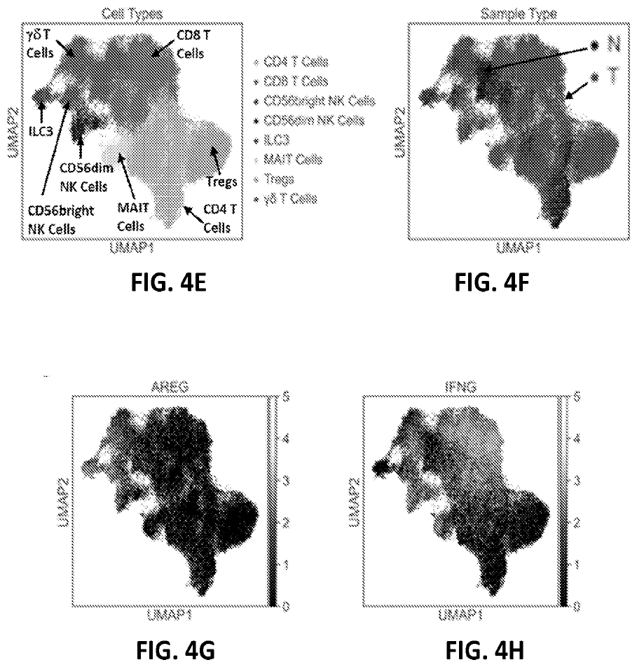

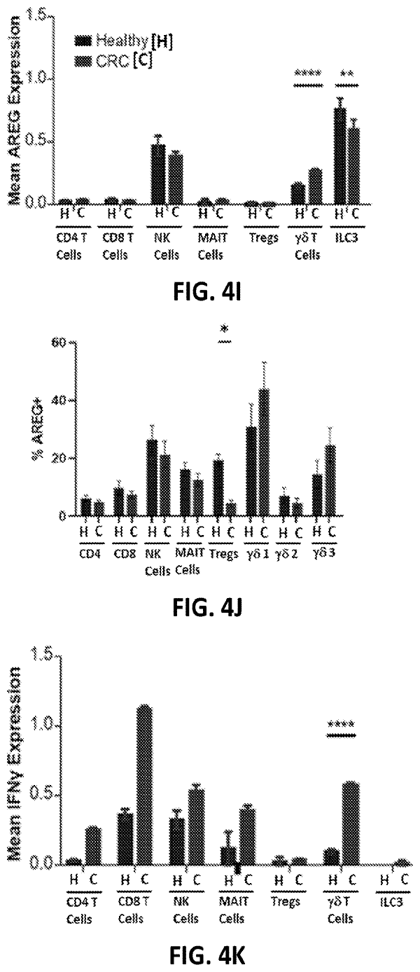

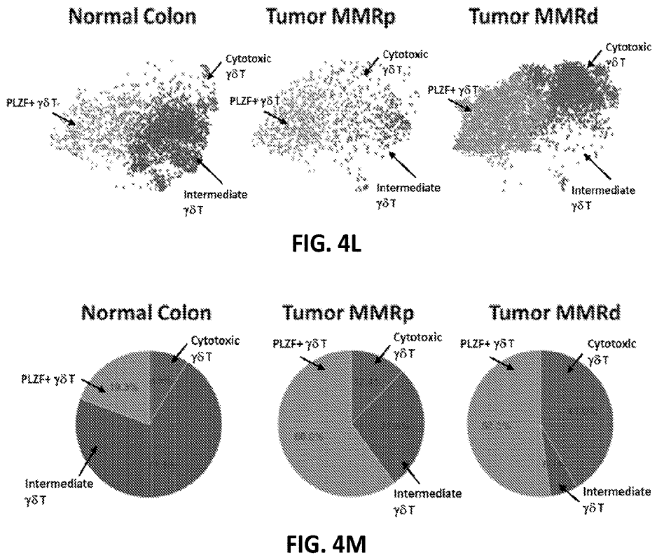

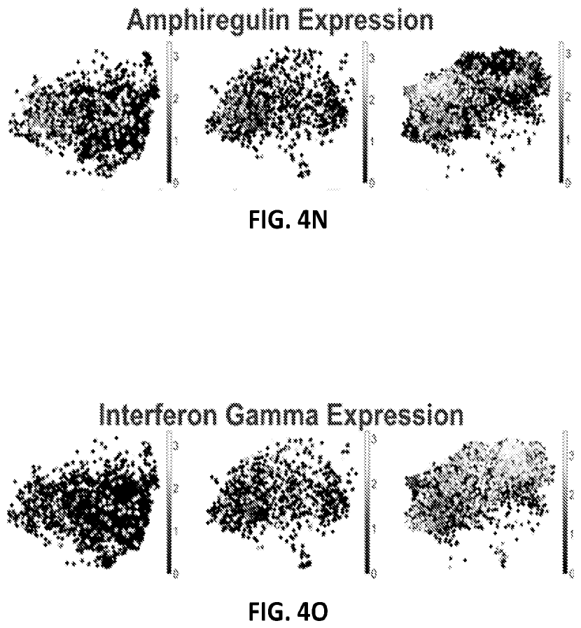

As demonstrated in FIGS. 4A-4O, CRC tumors are enriched in AREG, as compared to healthy tissue and endometrial tumors. Further, AREG is associated with MSS tumors and a wound healing molecular phenotype (CMS2). The skewing of Vδ1 T cells to an AREG producing wound healing phenotype is found to be associated with a subset of CRC patients, MMRp/MSS, who have poorer disease outcomes. In vitro data shows that AREG producing γδ T cells contribute to tumor growth. The effect of AREG producing Vδ1 T cells on wound healing was inhibited by cetuximab, which has been used as a therapy in a subset of CRC patients (Bridgewater et al., Lancet Oncol. 21, 398-411 (2020); Jonker et al., N. Engl. J. Med. 357, 2040-2048 (2007)), and blocking IL1b signaling also reduced the ability of Gen 1 cells to assist in wound healing. This demonstrates a tissue surveillance function for AREG producing γδ T cells.

MMRp patients generally respond poorly to immunotherapy. It is possible that, in MMRp CRC, Vδ1 T cells provide tumor growth factors (e.g., AREG) while also failing to provide an early source of IFNγ to promote a robust adaptive response (Larson et al., Nature (2022), doi:10.1038/s41586-022-04585-5). Using Gen 2 cells, the TME could be altered sufficiently to allow immune activation and better response to therapies.

As described in Example 5, in normal colon, AREG is produced by NK cells, ILC3s and γδ T cells. In normal colon, γδ T cells maintain an intermediate phenotype, indicating a potential to maintain and repair tissue integrity with an AREG based response, while also poised to respond to infection or tumors with an IFNγ based response. However, in malignancy, γδ T cells upregulated AREG production, particularly in MMRp tumors. In contrast, in MMRd tumors, there is both an AREG+ population and a significant emergence of cytotoxic γδ T cells. Interestingly, the γδ T cell population of the intestine also changes dramatically during colitis. Pathological inflammation of the gut resulted in the loss of AREG producing γδ T cells and an expansion of pro-inflammatory γδ T cells (Mayassi et al., Cell 176, 967-981.e19 (2019)). Similarly, in lung infection, AREG produced by γδ T cells is required for tissue repair and resolution of inflammation (Guo et al., Immunity 49, 531-544.e6 (2018)); however, some bacteria use this process as an immune deviation approach to allow colonization (Agaronyan et al., Immunity 1-17 (2022), doi:10.1016/j.immuni.2022.04.001). Furthermore, the lung microbiota has been shown to activate Vg6+Vδ1+ AREG producing γδ T cells in mice, which promote tumor development through the recruitment of neutrophils and induction of tumor cell proliferation (Jin et al., Cell 176, 998-1013.e16 (2019)).

The ability to identifying the two functional Vδ1 subsets by surface markers is useful in cell therapy and as a biomarker. As shown herein, cytotoxic Vδ1 cells were identifiable by the expression of TIGIT, a NK cell related inhibitory receptor (Yu et al., Nat. Immunol. 10, 48-57 (2009); Marin-Acevedo et al., J. Hematol. Oncol. 11, 39 (2018)). These studies indicate that current checkpoint therapies probably do not elicit much effect on γδ T cells, since they lack the expression of PD-1 or CTLA-4 in human colon and endometrial cancer. However, TIGIT appears to be an important checkpoint for innate T cells and NK cells and may provide a critical target for combination therapies using γδ T or NK cells to boost all anti-tumor immunity. TIGIT is becoming increasingly attractive checkpoint inhibitor target, and is in active clinical trials (Ge et al., Front. Immunol. 12, (2021); Harjunpää et al., Clin. Exp. Immunol. 200, 108-119 (2020)). Meanwhile, NKp80 (KLRF1) was associated with cells producing AREG. NKp80 has previously been shown to be differentially regulated between human Vδ1 and Vδ2 T cells; however, its function in NK and CD8 T cells has been associated with cytotoxicity and effector memory phenotypes (Pizzolato et al., Proc. Natl. Acad. Sci. 2019, 201818488 (2019); Freud et al., Cell Rep. 16, 379-391 (2016); Vitale et al., Eur. J. Immunol. 31, 233-42 (2001); Kuttruff et al., Blood 113, 358-69 (2009)). Data described hereinafter show that NKp80 is a marker of wound healing γδ T cells.

The present disclosure uses a published protocol to expand Vδ1 T cells (Almeida et al., Clin. Cancer Res. 22, 5795-5804 (2016); Di Lorenzo et al., Cancer Immunol. Res. 7, 552-558 (2019)) and shows that Vδ1 T cells expanded in this way are a mixed population which may not be ideal for cellular therapy against solid tumors, particularly CRC or lung cancer, due to AREG production. The present disclosure shows that Val T cells produce AREG in response to tumors in a wound healing assay, leading to increased tumor cell growth and repair of the “wound”. Notably this process could be ameliorated by the addition of Cetuximab, which has previously been associated with high efficacy in CMS2 colon cancer, where AREG expression is highest (Lenz et al., J. Clin. Oncol. 37, 1876-1885 (2019)). Importantly, in vivo, the TME in humanized mice also strongly induced AREG production by adoptively transferred Vδ1 T cells. Thus, elimination of this feature of Vδ1 T cells may be essential for γδ T cell therapy against solid tumors, especially where AREG production is associated with negative prognosis. Deconstructing the Gen 1 cytokine cocktail, it was identified that AREG production was primarily driven by IL-1β treatment, while IL-15 significantly reduced AREG production and induced IFNγ. Based on this, the Gen 2 cocktail described here (e.g., the first medium and/or the second medium described here) reduced AREG, enhanced cytotoxic function and boosted cellular metabolism. The addition of IL-18 possibly enhanced metabolic function as seen in memory NK cells and through suppression of TOX in tumor infiltrating CD8 T cells (Terren et al., Sci. Rep. 11, 6472 (2021); Cooper et al., Proc. Natl. Acad. Sci. 106, 1915-1919 (2009); Zhou et al., Nature 583, 609-614 (2020)). A key question in the field is how to generate a cellular therapy that can withstand the TME while maintaining effector functions. Exhaustion of CD8 T cells and NK cells is typically underpinned by metabolic deficits (Scharping et al., Immunity 45, 374-388 (2016); Yu et al., Nat. Immunol. (2020) doi:10.1038/s41590-020-0793-3; Qiu et al., Cell Rep. 27, 2063-2074.e5 (2019); Buck et al., J. Exp. Med. 212, 1345-1360 (2015); Michelet et al., Nat. Immunol. 19, 1330-1340 (2018)). The present disclosure (e.g., in Example 8) shows that the enhanced metabolic fitness in Gen 2 Vδ1 T cells can be maintained in vitro and in vivo in the TME. Mitochondrial fitness is essential for maintaining anti-tumor immunity in the tumor microenvironment and also thought to be associated with longevity. In acidic and hypoxic conditions, similar to that of a human tumor, Gen 2 cells did not upregulate mitochondrial ROS production and maintained their effector functions. In human patient derived xenografts of colon cancer, Gen 2 cells showed improved survival in the tumor and tumor control compared to Gen 1 cells after 7 days of treatment in large established tumors. Tumor infiltrating Gen 2 cells maintained their enhanced metabolic profile, and importantly Gen 2 cells did not produce AREG in the tumor, or in any tissue. As the TME and lung induced AREG from Gen 1 cells, it suggests that Gen 2 were also exposed to AREG inducing conditions in vivo, but that this cellular program was stably silenced during expansion. This was also accompanied by increased intra-tumoral production of IFNγ, contributing to overall better tumor control.

The present study shows that despite their strong association with positive prognosis in cancer, 78 T cells can be skewed toward pro-tumor function in the certain microenvironments. Resident γδ T cells in mucosal tissues play an important role in tissue maintenance and repair in homeostasis and infection, including the lung, oral mucosa and the gut (Suzuki et al., Immunol. Rev. 298, 198-217 (2020); Krishnan et al., Proc. Natl. Acad. Sci. 115, 10738-10743 (2018); Guo et al., Immunity 49, 531-544.e6 (2018); Zaiss et al., Immunity 42, 216-226 (2015)). In mice, this pro-tumor function is associated with IL-17 producing γδ T cells (Raverdeau et al., Clin. Transl. Immunol. 8, 1-15 (2019)); however, human γδ T cells produce little or no IL-17, therefore AREG may represent a better correlate between murine and human studies. This pro-tumor phenotype can be overcome during ex vivo expansion, producing only anti-tumor Vδ1 T cells. This has important implications for all cellular therapies for cancer as this pathway may be translatable to the CAR-T, CAR-NK, CAR-γδ T cells and TCR based therapeutics. This approach can provide a novel therapy for colon cancer patients not usually selected for immunotherapy due to MSS status. Administration of an anti-tumor Vδ1 T cells, protected from polarization to AREG production and metabolic paralysis, could provide a kickstart to wider anti-tumor immune function and convert “cold” tumors to immunologically “hot” tumors.

The present disclosure profiles innate T cells in human cancer for the first time and compares these cells to NK cells and conventional T cells from mismatch repair proficient (MMRp) colorectal and endometrial tumors. In colorectal cancer (CRC), a population of Vδ1 T cells was identified that produces amphiregulin (AREG) in vitro and in vivo in response to colon tumor cells. Colon tissue resident γδ T cells play an important role in tissue homeostasis and as tumors develop, this process prevents an appropriate immune response, allowing for ‘wounds’ that never heal. This feature of γδ T cells, while important for homeostasis, may be detrimental for their use as an adoptive cell therapy. The expansion method described here polarizes human Vδ1 T cells to cytotoxic killer cells and away from the wound healing phenotype. This method produces long lived cytotoxic effector cells which do not upregulate AREG in the tumor microenvironment. This cytotoxic profile is underpinned by metabolic reprogramming, sustaining effector function and longevity. After adoptive transfer into humanized mice with CRC, these superior anti-tumor Vδ1 T cells show infiltration and survival in tumors and reduced tumor burden in established colon tumors. Thus, described here are metabolically enhanced Vδ1 T cells that are strong anti-tumor candidates for development of a universal off the shelf adoptive cell therapy.

### Vδ1 T Cells

Described here are γδ T cells, in particular, γδ T cells of Vδ1 subtype, which may be referred to herein interchangeably as Vδ1 T cells or Vδ1 cells or Vd1 T cells or Vd1 cells. Also described herein are populations of such cells. In some instances, the Vδ1 T cells expanded by the methods of the present disclosure are referred to herein as Generation 2 cells or Gen 2 cells or 2^{nd} generation cells or second generation cells. Vδ1 T cells generated and/or expanded by other methods (e.g., methods previously known in the art) may be referred to herein as Generation 1 cells or Gen 1 cells or 1st generation cells or first generation cells. Additionally, or in the alternative, Gen 1 cells can be a population of naturally occurring Vδ1 T cells. For example, Gen 1 cells can be a population of Vδ1 T cells isolated from a subject, such as from a human subject (e.g., Vδ1 T cells isolated from a human biological sample). Additionally, or in the alternative, Gen 1 cells can be a population of Vδ1 T cells before these are expanded by the methods described here. For example, Gen 1 cells can be a population of Vδ1 T cells isolated from a subject, such as a human (e.g., Vδ1 T cells isolated from human biological sample) that have not been subjected to the expansion methods described here.

As compared to a population of Gen 1 cells, a population of Gen 2 cells described here may have one or more of the following: higher expression of (e.g., higher percentage of cells in the population expressing) T cell immunoreceptors with Ig and ITIM domains (TIGIT); lower expression of (e.g., lower percentage of cells in the population expressing) NKp80; higher expression of (e.g., higher percentage of cells in the population expressing and/or secreting) interferon gamma (IFNγ); lower expression of (e.g., lower percentage of cells in the population expressing and/or secreting) amphiregulin (AREG); higher expression of (e.g., higher percentage of cells in the population expressing) one or more cytotoxic effector molecules, e.g., when co-incubated with cancer cells; lower expression of (e.g., lower percentage of cells in the population expressing) one or more inhibitory molecules (e.g., inhibitory checkpoint molecules); lower expression of (e.g., lower percentage of cells in the population expressing) one or more exhaustion markers; (e.g., inhibitory checkpoint molecules); higher mitochondrial mass; higher membrane potential; higher basal glycolysis; higher glycolytic capacity; higher basal oxidative phosphorylation; higher maximum respiratory rate; higher persistence or longevity; higher expression of MYC; higher expression of SLC7A1; and/or higher expression of SLC1A5. Additionally, or in the alternative, as compared to a population of Gen 1 cells, a population of Gen 2 cells may have higher purity, e.g., a higher percentage of Vδ1 T cells. For example, in a population of Gen 2 cells, more than 80% (e.g., more than 85%, more than 90%, more than 95%, more than 96%, more than 97%, more than 98%, more than 99%, or 100%) of the cells may be Vδ1 T cells.

### TIGIT Expression

The Vδ1 T cells of the present disclosure (e.g., Vδ1 T cells expanded by the methods described here) can express T cell immunoreceptors with Ig and ITIM domains (TIGIT) (Gene ID: 201633). For example, in a population of Vδ1 T cells described here, 20% to 100% (e.g., 25% to 95%, 30% to 90%, 35% to 85%, 40% to 80%, 45% to 75%, 50% to 70%, or 55% to 65%) of the cells, such as, 20% or more (e.g., 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%) cells can express TIGIT. Exemplary sequence of human TIGIT is provided at NCBI Reference Sequence: NP_776160.2 (encoded by NM_173799.4).

In some instances, a population of Vδ1 T cells expanded by the methods described here (e.g., Gen 2 cells) comprises a higher frequency of cells expressing TIGIT (e.g., a higher frequency of TIGIT+ cells), as compared to a reference population of Vδ1 T cells. A reference population of Vδ1 T cells can be a population of Vδ1 T cells that have not been expanded and/or generated and/or obtained by the methods described here. For example, a reference population of Vδ1 T cells can be a population of Vδ1 T cells that have been expanded and/or generated and/or obtained by method previously known in the art. Thus, a reference population of Vδ1 T cells can be a population of Gen 1 cells. Additionally, or in the alternative, a reference population of Vδ1 T cells can be a population of Vδ1 T cells before these are expanded by the methods described here. For example, a reference population of Vδ1 T cells can be a population of Vδ1 T cells isolated from a subject, such as a human (e.g., Vδ1 T cells isolated from human biological sample) that have not been subjected to the expansion methods described here. In some instances, the frequency of TIGIT+ cells in a population of Vδ1 T cells expanded by the methods described here is 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to the frequency of TIGIT+ cells in a reference population of Vδ1 T cells.

### NKp80 Expression

The Vδ1 T cells of the present disclosure (e.g., Vδ1 T cells expanded by the methods described here) may not express the C-type lectin-like surface activating receptor, NKp80 (Gene ID: 51348). For example, in a population of Vδ1 T cells described here, 20% to 100% (e.g., 25% to 95%, 30% to 90%, 35% to 85%, 40% to 80%, 45% to 75%, 50% to 70%, or 55% to 65%) of the cells, such as, 20% or more (e.g., 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%) cells may not express NKp80 (e.g., may be NKp80 negative or NKp80−). Exemplary sequence of human NKp80 isoform 1 is provided at NCBI Reference Sequence: NP_057607.1 (encoded by NM 016523.3). Exemplary sequence of human NKp80 isoform KLRF1-s is provided at NCBI Reference Sequence: NP_001278751.1 (encoded by NM_001291822.2). Exemplary sequence of human NKp80 isoform KLRF1-s3 is provided at NCBI Reference Sequence: NP_001278752.1 (encoded by NM_001291823.2). Exemplary sequence of human NKp80 isoform 2 is provided at NCBI Reference Sequence: NP_001353463.1 (encoded by NM_001366534.1).

In some instances, a population of Vδ1 T cells expanded by the methods described here (e.g., Gen 2 cells) comprises a higher frequency of cells not expressing NKp80 (e.g., a higher frequency of NKp80− cells), as compared to a reference population of Vδ1 T cells. For example, the frequency of NKp80− cells in a population of Vδ1 T cells expanded by the methods described here can be 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to the frequency of NKp80− cells in a reference population of Vδ1 T cells.

In some instances, in a population of Vδ1 T cells described here, less than 40% (e.g., less than 35%, less than 30%, less than 25%, less than 20%, less than 15%, less than 10%, less than 5%, less than 3%, less than 2%, or less than 1%) of the cells, such as, about 0% to 40% (e.g., about 0% to 5%, about 5% to 10%, about 10% to 15%, about 15% to 20%, about 20% to 25%, about 25% to 30%, about 30% to 35%, or about 35% to 40%) of the cells express NKp80 (e.g., may be NKp80+).

### IFNγ Expression

The Vδ1 T cells of the present disclosure (e.g., Vδ1 T cells expanded by the methods described here) can express and/or secrete interferon gamma (IFNγ) (Gene ID: 3458). For example, when activated (e.g., following stimulation with cytokines or TCR agonists or mitogens (e.g., PMA and/or ionomycin, combinations of IL-12/IL-15/IL-18), co-incubation with cancer cells, etc.), the Vδ1 T cells described here can express and/or secrete IFNγ. In some instances, in a population of Vδ1 T cells described here, 20% to 100% (e.g., 25% to 95%, 30% to 90%, 35% to 85%, 40% to 80%, 45% to 75%, 50% to 70%, or 55% to 65%) of the cells, such as, 20% or more (e.g., 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%) cells can express and/or secrete IFNγ. Exemplary sequence of human IFNγ precursor is provided at NCBI Reference Sequence: NP_000610.2 (encoded by NM_000619.3).

In some instances, a population of Vδ1 T cells expanded by the methods described here (e.g., Gen 2 cells) comprises a higher frequency of cells expressing and/or secreting IFNγ, as compared to a reference population of Vδ1 T cells. For example, the frequency of cells expressing and/or secreting IFNγ in a population of Vδ1 T cells expanded by the methods described here can be 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to the frequency of cells expressing and/or secreting IFNγ in a reference population of Vδ1 T cells.

### AREG Expression

The Vδ1 T cells of the present disclosure (e.g., Vδ1 T cells expanded by the methods described here) may not express and/or secrete amphiregulin (AREG) (Gene ID: 374). For example, when activated (e.g., in the presence of damaged cells, when engaged by EGFR on tumor cells or damaged cells, following stimulation with TCR agonists or mitogens or cytokines (e.g., PMA and/or ionomycin), co-incubation with cancer cells, etc.), the Vδ1 T cells described here may not express and/or secrete AREG. In some instances, in a population of Vδ1 T cells described here, 20% to 100% (e.g., 25% to 95%, 30% to 90%, 35% to 85%, 40% to 80%, 45% to 75%, 50% to 70%, or 55% to 65%) of the cells, such as, 20% or more (e.g., 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%) cells do not express and/or secrete AREG. Exemplary sequence of human AREG preproprotein is provided at NCBI Reference Sequence: NP_001648.1 (encoded by NM_001657.4).

In some instances, in a population of Vδ1 T cells described here, less than 40% (e.g., less than 35%, less than 30%, less than 25%, less than 20%, less than 15%, less than 10%, less than 5%, less than 3%, less than 2%, or less than 1%) of the cells, such as, about 0% to 40% (e.g., about 0% to 5%, about 5% to 10%, about 10% to 15%, about 15% to 20%, about 20% to 25%, about 25% to 30%, about 30% to 35%, or about 35% to 40%) of the cells express and/or secrete AREG.

In some instances, a population of Vδ1 T cells expanded by the methods described here (e.g., Gen 2 cells) comprises a higher frequency cells that do not express and/or secrete AREG, as compared to a reference population of Vδ1 T cells. For example, in a population of Vδ1 T cells expanded by the methods described here, the frequency of cells not expressing and/or secreting AREG can be 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to the frequency of cells not expressing and/or secreting AREG in a reference population of Vδ1 T cells.

### Cytotoxicity

The Vδ1 T cells of the present disclosure (e.g., Vδ1 T cells expanded by the methods described here) can be cytotoxic in nature. For example, the Vδ1 T cells described here can show cytotoxicity when co-incubated with cancer cells. Cytotoxicity exhibited by the Vδ1 T cells can include, without limitation, one or more of: killing of cancer cells, induction of caspase signaling in tumor cells, degranulation of cytotoxic machinery in tumor cells, induction of TRAIL, inhibition of cancer cell proliferation, inhibition of cancer cell growth, inhibition of cancer cell migration, inhibition of wound closing and/or wound healing (e.g., in a scratch assay), inhibition of tumor growth (e.g., when the Vδ1 T cells are administered to a subject with tumor), and expression of one or more cytotoxic effector molecules. In some instances, a cytotoxicity assay is used to measure killing of cancer cells by Vδ1 T cells, e.g., by co-culturing Vδ1 T cells with tumor cells and measuring tumor cell death by flow cytometry. Additionally, or in the alternative, degranulation assays (e.g., using LAMP1/CD107a) can be used to measure cytotoxicity of Vδ1 T cells in the presence of tumor cells.

In a population of Vδ1 T cells described here, 20% to 100% (e.g., 25% to 95%, 30% to 90%, 35% to 85%, 40% to 80%, 45% to 75%, 50% to 70%, or 55% to 65%) of the cells, such as, 20% or more (e.g., 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%) cells can be cytotoxic. For example, 20% to 100% cells in a population of Vδ1 T cells can show cytotoxicity when co-incubated with cancer cells.

A population of Vδ1 T cells expanded by the methods described here (e.g., Gen 2 cells) can comprise a higher frequency of cytotoxic cells, as compared to a reference population of Vδ1 T cells. For example, the frequency of cytotoxic cells in a population of Vδ1 T cells expanded by the methods described here can be 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to the frequency of cytotoxic cells in a reference population of Vδ1 T cells. In some instances, following co-incubation with cancer cells, the frequency of cytotoxic cells in a population of Vδ1 T cells described here is 1.5-fold or more, as compared to the frequency of cytotoxic cells in a reference population of Vδ1 T cells.

Cytotoxicity exhibited by cells in a population of Vδ1 T cells expanded by the methods described here can be higher, as compared to cytotoxicity exhibited by a reference population of Vδ1 T cells. For example, cytotoxicity exhibited by cells in a population of Vδ1 T cells (expanded by the methods described here) can be 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to cytotoxicity exhibited by a reference population of Vδ1 T cells.

### Cytotoxic Effector Molecules

In some instances, the Vδ1 T cells described here can express one or more cytotoxic effector molecules, including, but not limited to, granzyme K (GZMK), granzyme B (GZMB), perforin (PRF1), IFNγ (IFNG), CD107a, TNFα, RANTES (CCL5), granzyme K (gzmk), TRAIL, FAS-FAS Ligand, and granulysin (GNLY). In some instances, as compared to Gen 1 cells, the V51 T cells described here (e.g., Gen 2 cells) can have higher expression level for one or more cytotoxic effector molecules. In a population of Vδ1 T cells described here, 20% to 100% (e.g., 25% to 95%, 30% to 90%, 35% to 85%, 40% to 80%, 45% to 75%, 50% to 70%, or 55% to 65%) of the cells, such as, 20% or more (e.g., 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%) of the cells can express (e.g., high levels of) one or more cytotoxic effector molecules. For example, 20% to 100% cells in a population of Vδ1 T cells can express one or more cytotoxic effector molecules when co-incubated with cancer cells.

In some instances, a population of Vδ1 T cells expanded by the methods described here (e.g., Gen 2 cells) can express higher levels of one or more cytotoxic effector molecules, as compared to a reference population of Vδ1 T cells. For example, a population of Vδ1 T cells (expanded by the methods described here) can express one or more cytotoxic effector molecules at a level that is 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to the cytotoxic effector molecules expressed by a reference population of Vδ1 T cells.

In some instances, a population of Vδ1 T cells expanded by the methods described here can comprise a higher frequency of cells that express one or more cytotoxic effector molecules, as compared to a reference population of Vδ1 T cells. For example, in a population of Vδ1 T cells (expanded by the methods described here), the frequency of cells expressing cytotoxic effector molecule can be 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to the frequency of cells expressing cytotoxic effector molecule in a reference population of Vδ1 T cells.

### Inhibitory Molecules

The Vδ1 T cells described here have low or no detectable expression for one or more inhibitory molecules. In some instances, as compared to Gen 1 cells, the Vδ1 T cells described here (e.g., Gen 2 cells) can have lower expression level for one or more inhibitory molecules. As used herein, an “inhibitory molecule” may refer to a molecule that inhibits the activation of immune response. For example, an inhibitory molecule may refer to an inhibitory checkpoint molecule that inhibits immune activation, maintains immune homeostasis and/or prevents autoimmunity. Additionally, or in the alternative, an inhibitory molecule may refer to an inhibitory checkpoint molecule that inhibits anti-tumor immune response. Additionally, or in the alternative, an inhibitory molecule may refer to a molecule that inhibits activation signaling, TCR signaling, and/or cytokine production. Inhibitory molecules can include, without limitation, PD1 (PDCD1), CTLA4, TIM3 (HAVCR2), LAG3, AREG, certain NK receptors, TIGIT and other surface molecules with immunoreceptor tyrosine-based inhibitory motifs (ITIMs) present in their cytoplasmic domains.

In a population of Vδ1 T cells described here, 20% to 100% (e.g., 25% to 95%, 30% to 90%, 35% to 85%, 40% to 80%, 45% to 75%, 50% to 70%, or 55% to 65%) of the cells, such as, 20% or more (e.g., 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%) of the cells can have low expression levels (or no detectable expression) for one or more inhibitory molecules.

In some instances, a population of Vδ1 T cells expanded by the methods described here (e.g., Gen 2 cells) can express lower levels of one or more inhibitory molecules, as compared to a reference population of Vδ1 T cells. For example, a population of V51 T cells (expanded by the methods described here) can express one or more inhibitory molecules at a level that is 0.75-fold or lower (e.g., 0.7-fold or lower, 0.65-fold or lower, 0.6-fold or lower, 0.55-fold or lower, 0.5-fold or lower, 0.45-fold or lower, 0.4-fold or lower, 0.35-fold or lower, 0.3-fold or lower, 0.25-fold or lower, 0.2-fold or lower, 0.15-fold or lower, 0.1-fold or lower, 0.075-fold or lower, 0.05-fold or lower, 0.025-fold or lower, 0.01-fold or lower, 0.005-fold or lower, or 0.001-fold or lower), as compared to the inhibitory molecules expressed by a reference population of Vδ1 T cells.

In some instances, a population of Vδ1 T cells expanded by the methods described here can comprise a lower frequency of cells that express one or more inhibitory molecules, as compared to a reference population of Vδ1 T cells. For example, in a population of Vδ1 T cells (expanded by the methods described here), the frequency of cells expressing inhibitory molecules can be 0.75-fold or lower (e.g., 0.7-fold or lower, 0.65-fold or lower, 0.6-fold or lower, 0.55-fold or lower, 0.5-fold or lower, 0.45-fold or lower, 0.4-fold or lower, 0.35-fold or lower, 0.3-fold or lower, 0.25-fold or lower, 0.2-fold or lower, 0.15-fold or lower, 0.1-fold or lower, 0.075-fold or lower, 0.05-fold or lower, 0.025-fold or lower, 0.01-fold or lower, 0.005-fold or lower, or 0.001-fold or lower), as compared to the frequency of cells expressing inhibitory molecules in a reference population of Vδ1 T cells.

### Metabolic Activity

The Vδ1 T cells of the present disclosure (e.g., V51 T cells expanded by the methods described here) can be metabolically active and/or fit. Metabolically active and/or fit cells can withstand activation and/or harsh conditions (e.g., hypoxic condition and/or acidic condition) without becoming dysfunctional or without dying. Additionally, or in the alternative, metabolically active and/or fit cells can engage in energy pathways or metabolic pathways, including, but not limited to, glycolysis and oxidative phosphorylation pathway (OXPHOS). Thus, metabolically active and/or fit cells can be energetic cells, and not quiescent cells. In some instances, the Vδ1 T cells of the present disclosure are metabolically active and/or fit, as assessed, based on one or more of: mitochondrial mass, membrane potential, basal glycolysis, glycolytic capacity, basal oxidative phosphorylation, maximum respiratory rate, persistence or longevity, expression of MYC, expression of SLC7A1, and expression of SLC1A5. For example, in a population of Vδ1 T cells described here, 20% to 100% (e.g., 25% to 95%, 30% to 90%, 35% to 85%, 40% to 80%, 45% to 75%, 50% to 70%, or 55% to 65%) of the cells, such as, 20% or more (e.g., 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%) of cells can be metabolically active. In some instances, Vδ1 T cells described here are metabolically active even under hypoxic and/or acidic conditions. For example, in a population of Vδ1 T cells described here, 20% to 100% of cells can be metabolically active even under hypoxic and/or acidic conditions.

Hypoxic conditions can comprise exposing the cells to less than 5% (e.g., less than 4.5%, less than 4%, less than 3.5%, less than 3%, less than 2.5%, less than 2%, less than 1.5%, less than 1%, less than 0.5%, less than 0.4%, less than 0.3%, or less than 0.2%) O2, such as, about 0.1% to 4.5% O2 (e.g., 0.2% to 0.8% O2, 0.3% to 0.7% O2, 0.4% to 0.6% O2, 0.6% to 0.8% O2, 0.8% to 1% O2, 1% to 1.5% O2, 1.5% to 2.5% O2, 2.5% to 3.5% O2, or 3.5% to 4.5% O2; such as, 0.1% O2, 0.2% O2, 0.3% O2, 0.4% O2, 0.5% O2, 0.6% O2, 0.7% O2, 0.8% O2, 0.9% O2, 1% O2, 1.5% O2, 2% O2, 2.5% O2, 3% O2, 3.5% O2, 4% O2, or 4.5% O2).

Acidic conditions can comprise exposing the cells to 0-40 mM (e.g., 0-5 mM, 0-10 mM, 0-15 mM, 0-20 mM, 0-25 mM, 0-30 mM, 0-35 mM; such as, 5 mM, 10 mM, 15 mM, 20 mM, 25 mM, 30 mM, 35 mM, or 40 mM) lactic acid. Additionally, or in the alternative, acidic conditions can comprise exposing the cells to a pH of less than 7.0 (e.g., less than 6.9, less than 6.8, less than 6.7, less than 6.6, less than 6.5, less than 6.4, less than 6.3, less than 6.2, less than 6.1, or less than 6.0), such as, a pH of 6 to 7 (e.g., a pH of 6 to 6.1, a pH of 6.1 to 6.2, a pH of 6.2 to 6.3, a pH of 6.3 to 6.4, a pH of 6.4 to 6.5, a pH of 6.5 to 6.6, a pH of 6.6 to 6.7, a pH of 6.7 to 6.8, a pH of 6.8 to 6.9, or a pH of 6.9 to 7).

In some instances, a population of Vδ1 T cells described here can have improved metabolic activity and/or improved metabolic fitness, as compared to a reference population of Vδ1 T cells. For example, a population of Vδ1 T cells expanded by the methods described here (e.g., Gen 2 cells) can comprise a higher frequency of metabolically active cells, as compared to a reference population of Vδ1 T cells (e.g., a population of Gen 1 cells). In particular, in a population of Vδ1 T cells (expanded by the methods described here), the frequency of metabolically active cells can be 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to the frequency of metabolically active cells in a reference population of Vδ1 T cells.

In some instances, as compared to a reference population of Vδ1 T cells (e.g., a population of Gen 1 cells), a population of Vδ1 T cells expanded by the methods described here (e.g., Gen 2 cells) can exhibit one or more of: higher mitochondrial mass, higher membrane potential, higher basal glycolysis, higher glycolytic capacity, higher basal oxidative phosphorylation, higher maximum respiratory rate, higher persistence or longevity, higher expression of MYC, higher expression of SLC7A1, and higher expression of SLC1A5. For example, a population of Vδ1 T cells (expanded by the methods described here) can have one or more of—mitochondrial mass, membrane potential, basal glycolysis, glycolytic capacity, basal oxidative phosphorylation, maximum respiratory rate, persistence or longevity, expression of MYC, expression of SLC7A, and expression of SLC1A5—at a level that is 1.5-fold or higher (e.g., 2.0-fold or higher, 2.5-fold or higher, 3.0-fold or higher, 3.5-fold or higher, 4.0-fold or higher, 4.5-fold or higher, 5.0-fold or higher, 5.5-fold or higher, 6.0-fold or higher, 6.5-fold or higher, 7.0-fold or higher, 7.5-fold or higher, 8.0-fold or higher, 8.5-fold or higher, 9.0-fold or higher, 9.5-fold or higher, 10.0-fold or higher, 15-fold or higher, 20.0-fold or higher, 25-fold or higher, 30.0-fold or higher, 35-fold or higher, 40.0-fold or higher, 45-fold or higher, 50.0-fold or higher, 55-fold or higher, 60.0-fold or higher, 65-fold or higher, 70.0-fold or higher, 75-fold or higher, 80.0-fold or higher, 85-fold or higher, 90.0-fold or higher, 95-fold or higher, or 100-fold or higher), as compared to that exhibited by a reference population of Vδ1 T cells.

Additionally, or in the alternative, as compared to a reference population of Vδ1 T cells (e.g., a population of Gen 1 cells produced by a method as described in Almeida et al. (Clin. Cancer Res. 22, 5795-5804 (2016) and/or Di Lorenzo et al. (Cancer Immunol. Res. 7, 552-558 (2019)), a population of Vδ1 T cells expanded by the methods described here (e.g., Gen 2 cells) can have lower expression of one or more exhaustion markers, including, but not limited to, PD-1, KLRG-1, LAG-3, and TIM-3, CTLA4, TOX, NKp80. In some instances, the Vδ1 T cells of the present disclosure (e.g., Gen 2 cells) have no detectable expression for one or more exhaustion markers. An “exhaustion marker” may refer to a cell surface marker that is over-expressed on an exhausted T cell, as compared to an active T cell. An exhausted T cell can exhibit one or more of: T cell dysfunction resulting from chronic stimulation; overexpression of one or more inhibitory checkpoint molecules; impairment in the ability to express/secrete one or more cytotoxic effector molecules. Exhaustion may occur in the tumor microenvironment where T cells may suffer a loss of their cytotoxic function and/or become ineffective in their ability to kill cancer cells.

For example, in a population of Vδ1 T cells described here, 20% to 100% (e.g., 25% to 95%, 30% to 90%, 35% to 85%, 40% to 80%, 45% to 75%, 50% to 70%, or 55% to 65%) of the cells, such as, 20% or more (e.g., 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, or 100%) of the cells can have low expression level (or no detectable expression level) for one or more exhaustion markers.

In some instances, a population of Vδ1 T cells expanded by the methods described here (e.g., Gen 2 cells) can express lower levels of one or more exhaustion markers, as compared to a reference population of Vδ1 T cells. For example, a population of Vδ1 T cells (expanded by the methods described here) can express one or more exhaustion markers at a level that is 0.75-fold or lower (e.g., 0.7-fold or lower, 0.65-fold or lower, 0.6-fold or lower, 0.55-fold or lower, 0.5-fold or lower, 0.45-fold or lower, 0.4-fold or lower, 0.35-fold or lower, 0.3-fold or lower, 0.25-fold or lower, 0.2-fold or lower, 0.15-fold or lower, 0.1-fold or lower, 0.075-fold or lower, 0.05-fold or lower, 0.025-fold or lower, 0.01-fold or lower, 0.005-fold or lower, or 0.001-fold or lower), as compared to the exhaustion markers expressed by a reference population of Vδ1 T cells.

In some instances, a population of Vδ1 T cells expanded by the methods described here can comprise a lower frequency of cells that express one or more exhaustion markers, as compared to a reference population of Vδ1 T cells. For example, in a population of Vδ1 T cells (expanded by the methods described here), the frequency of cells expressing exhaustion markers can be 0.75-fold or lower (e.g., 0.7-fold or lower, 0.65-fold or lower, 0.6-fold or lower, 0.55-fold or lower, 0.5-fold or lower, 0.45-fold or lower, 0.4-fold or lower, 0.35-fold or lower, 0.3-fold or lower, 0.25-fold or lower, 0.2-fold or lower, 0.15-fold or lower, 0.1-fold or lower, 0.075-fold or lower, 0.05-fold or lower, 0.025-fold or lower, 0.01-fold or lower, 0.005-fold or lower, or 0.001-fold or lower), as compared to the frequency of cells expressing exhaustion markers in a reference population of Vδ1 T cells.

Additionally, or in the alternative, improved metabolic activity exhibited by the Vδ1 T cells can include maintaining cytotoxicity (cytotoxicity exhibited by Vδ1 T cells is described in detail in the foregoing section) under hypoxic and/or acidic conditions. For example, in a population of Vδ1 T cells described here, 20% to 100% of cells can exhibit cytotoxicity under hypoxic and/or acidic conditions. In some instances, there is less than 50% (e.g., less than 45%, less than 40%, less than 35%, less than 30%, less than 25%, less than 20%, less than 15%, less than 10%, less than 5%, or less than 1%) decrease in cytotoxicity exhibited by the Vδ1 T cells under hypoxic and/or acidic conditions, as compared to cytotoxicity exhibited by the Vδ1 T cells under normal conditions. In particular instances, Vδ1 T cells described here show no decrease in cytotoxicity under hypoxic and/or acidic conditions, as compared to cytotoxicity of the Vδ1 T cells under normal conditions. Normal conditions can comprise a normal growing condition for the cells, such as about 5% to 10% O2 and/or a pH of 7.0 to 7.4.

Additionally, or in the alternative, improved metabolic activity exhibited by Vδ1 T cells described here can include no substantial increase in mitochondrial ROS production by the cells under hypoxic and/or acidic conditions. For example, there can be less than 50% (e.g., less than 45%, less than 40%, less than 35%, less than 30%, less than 25%, less than 20%, less than 15%, less than 10%, less than 5%, or less than 1%) increase in ROS production by the Vδ1 T cells under hypoxic and/or acidic conditions, as compared to mitochondrial ROS production by the Vδ1 T cells under normal conditions. In particular instances, Vδ1 T cells described here show no increase in mitochondrial ROS production under hypoxic and/or acidic conditions, as compared to mitochondrial ROS production by the Vδ1 T cells under normal conditions. In some instances, the level of mitochondrial ROS produced by a population of Vδ1 T cells (expanded by the methods described here) is 0.75-fold or lower (e.g., 0.7-fold or lower, 0.65-fold or lower, 0.6-fold or lower, 0.55-fold or lower, 0.5-fold or lower, 0.45-fold or lower, 0.4-fold or lower, 0.35-fold or lower, 0.3-fold or lower, 0.25-fold or lower, 0.2-fold or lower, 0.15-fold or lower, 0.1-fold or lower, 0.075-fold or lower, 0.05-fold or lower, 0.025-fold or lower, 0.01-fold or lower, 0.005-fold or lower, or 0.001-fold or lower), as compared to the level of mitochondrial ROS produced by a reference population of Vδ1 T cells.

### Expansion of Vδ1 T Cells

Also described here are methods for expansion of Vδ1 T cells of the present disclosure. For expansion by the methods described here, Vδ1 T cells can be isolated from a biological sample of a human (e.g., from a biological sample obtained from a human). The biological sample can be blood (e.g., peripheral blood or cord blood) and/or bone marrow. In some instances, Vδ1 T cells are isolated from a population of peripheral blood mononuclear cells (PBMCs) that are isolated from the biological sample. The PBMCs can be isolated from the biological sample by density gradient centrifugation. For example, isolation of PBMCs from blood may comprise separation of the PBMCs by density gradient centrifugation (e.g., using FICOLL-PAQUE™ PLUS (GE Healthcare)) and/or removal of residual red blood cells by lysis (e.g., by adding red cell lysis solution). One or more depletion steps can then be used for depletion of monocytes, macrophages, and/or alpha beta T (αβ T) cells from the population of PBMCs. The one or more depletion steps may comprise adherence. For example, monocytes and/or macrophages can be removed from the population of PBMCs by adherence. Additionally, or in the alternative, the one or more depletion steps may comprise complement-dependent-cytotoxicity (CDC). For example, αβ T cells can be removed from the population of PBMCs by CDC. Additionally, or in the alternative, the one or more depletion steps may comprise negative selection, e.g., with a combination of antibodies directed to cell surface markers present on the cells negatively selected. Cell sorting, selection via negative magnetic immunoadherence and/or flow cytometry can be used for depletion of cells by negative selection methods. For example, αβ T cells can be removed from the population of PBMCs by negative selection, e.g., with a combination of antibodies directed to surface markers unique to the up T cells. One or more of these methods can be used for isolation of Vδ1 T cells from a biological sample.

Vδ1 T cells (e.g., Vδ1 T cells isolated from a biological sample) can be identified by one or more methods known in the art. In some instances, Vδ1 T cells are identified by immunophenotyping methods, e.g., by using antibodies directed to one or more cell surface markers that are present (e.g., exclusively present) on Vδ1 T cells. For example, a cell can be identified as a Vδ1 T cell by detecting the presence of one or more cell surface markers that are known to be present on Vδ1 T cells. Such cell surface markers may include, without limitation, one or more of TCR Vδ1, CD3, NKG2D, γδTCR, and TIGIT. Additionally, or in the alternative, Vδ1 T cells can be identified by immunophenotyping methods, e.g., by using antibodies directed to one or more cell surface markers that are not present on Vδ1 T cells. For example, a cell can be identified as a Vδ1 T cell by detecting the absence of one or more cell surface markers (e.g., cell surface markers that are known to be not present on Vδ1 T cells) on the cell. Such cell surface markers may include, without limitation, one or more of CD14, CD68, CD19, CD21, CD56, and CD4. The presence and/or absence of cell surface markers can be detected by one or more method known on the art, including, but not limited to immunofluorescence methods, such as, flow cytometry or immunofluorescence or PCR or qPCR (e.g., for detection of TCD Vδ1 genes (TRDV1)).

Vδ1 T cells isolated from a biological sample can be expanded by the methods described herein to obtain the Vδ1 T cells of the present disclosure (e.g., the Gen 2 cells). For expansion of the Vδ1 T cells, Vδ1 T cells isolated from a biological sample can be cultured in a first cell culture medium (e.g., a first medium) for about 10-20 days (e.g., for about 10-12 days, about 12-14 days, about 14-16 days, about 16-18 days, or about 18-20 days; such as, for about 14 or 15 days). For example, Vδ1 T cells isolated from a biological sample (e.g., a human biological sample) can be cultured in a first cell culture medium for about 2 weeks (e.g., for about 1-2 weeks, such as, about 1 week, 1.5 weeks, or 2 weeks). Vδ1 T cells isolated from a biological sample can then be cultured in a second cell culture medium (e.g., a second medium) for about 3-10 days (e.g., for about 3-5 days, about 5-7 days, about 7-8 days, or about 7-10 days; such as, for about 7 or 8 days). For example, Vδ1 T cells isolated from a biological sample (e.g., a human biological sample) can be cultured in a second cell culture medium for about 1 week (e.g., for about 0.5 week or 1 week. For example, Vδ1 T cells isolated from a human biological sample can be cultured in a first medium for about 10-20 days (e.g., about 2 weeks); then, the first medium can be replaced with a second medium; and the cells can be cultured in the second medium for another 3-10 days (e.g., one more week).

In some instances, the first medium comprises one, two, three, four, five, six, or all seven of: a human serum (or a serum alternative or serum replacement), glutamine, interleukin (IL)-1β, IL-4, IL-21, interferon gamma (IFNγ), and an anti-CD3 antibody (e.g., OKT3, UCHT1, HIT3a) or an antigen-binding fragment thereof. In some instances, the first medium can comprise a basal medium (e.g., CTS OPTMIZER T Cell Expansion SFM (GIBCO)) supplemented with one or more of (e.g., one, two, three, four, five, six, or all seven of): a human serum (or a serum alternative or serum replacement), glutamine, IL-1β, IL-4, IL-21, IFNγ, and an anti-CD3 antibody or an antigen-binding fragment thereof. In particular, the first medium can comprise one, two, three, four, five, six, or all seven of
about 10% to 10% (e.g., about 2% to 4%, about 4% to 6%, about 6% to 8%, or about 8% to 10%; such as, about 1%, 2%, 3%, 4%, 5%, 6%, 7%, 8%, 9%, or 10%) human serum (or a serum alternative or serum replacement);
about 1 mmol to 5 mmol (such as, about 1 mmol to 2 mmol, about 2 mmol to 3 mmol, about 3 mmol to 4 mmol, or about 4 mmol to 5 mmol (e.g., about 1 mmol, about 2 mmol, about 3 mmol, about 4 mmol, or about 5 mmol)) glutamine;
about 10 ng/ml to 20 ng/ml (such as, about 10 ng/ml to 12 ng/ml, about 12 ng/ml to 14 ng/ml, about 14 ng/ml to 16 ng/ml, about 16 ng/ml to 18 ng/ml, or about 18 ng/ml to 20 ng/ml (e.g., about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, or about 20 ng/ml)) IL-13;
about 50 ng/ml to 150 ng/ml (such as, about 50 ng/ml to 70 ng/ml, about 70 ng/ml to 90 ng/ml, about 90 ng/ml to 110 ng/ml, about 110 ng/ml to 130 ng/ml, or about 130 ng/ml to 150 ng/ml (e.g., about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, or about 150 ng/ml) IL-4;
about 1 ng/ml to 15 ng/ml (such as, about 1 ng/ml to 3 ng/ml, about 3 ng/ml to 5 ng/ml, about 5 ng/ml to 7 ng/ml, about 7 ng/ml to 9 ng/ml, about 9 ng/ml to 11 ng/ml, about 11 ng/ml to 13 ng/ml, or about 13 ng/ml to 15 ng/ml (e.g., about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, or about 15 ng/ml)) IL-21;
about 10 ng/ml to 150 ng/ml (such as, about 10 ng/ml to 30 ng/ml, about 30 ng/ml to 50 ng/ml, about 50 ng/ml to 70 ng/ml, about 70 ng/ml to 90 ng/ml, about 90 ng/ml to 110 ng/ml, about 110 ng/ml to 130 ng/ml, or about 130 ng/ml to 150 ng/ml (e.g., about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, or about 150 ng/ml)) IFNγ; and/or
about 10 ng/ml to 150 ng/ml (such as, about 10 ng/ml to 30 ng/ml, about 30 ng/ml to 50 ng/ml, about 50 ng/ml to 70 ng/ml, about 70 ng/ml to 90 ng/ml, about 90 ng/ml to 110 ng/ml, about 110 ng/ml to 130 ng/ml, or about 130 ng/ml to 150 ng/ml (e.g., about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, or about 150 ng/ml)) of an anti-CD3 antibody (e.g., OKT3, UCHT1, HIT3a) or an antigen-binding fragment thereof.

In some instances, the second medium comprises one, two, three, four, five, six, or all seven of: a human serum (or a serum alternative or serum replacement), glutamine, IL-15, IFNγ, IL18, an anti-CD2 antibody (e.g., RPA-2.10, TS2/18, AB75) or an antigen-binding fragment thereof, and an anti-CD3 antibody (e.g., OKT3, UCHT1, HIT3a) or an antigen-binding fragment thereof. In some instances, the second medium can comprise a basal medium (e.g., CTS OPTMIZER T Cell Expansion SFM (GIBCO)) supplemented with one or more of (e.g., one, two, three, four, five, six, or all seven of): a human serum (or a serum alternative or serum replacement), glutamine, IL-15, IFNγ, IL18, an anti-CD2 antibody or an antigen-binding fragment thereof, and an anti-CD3 antibody or an antigen-binding fragment thereof. In particular, the second medium can comprise one, two, three, four, five, six, or all seven of:
about 0.1% to 5% (e.g., about 0.1% to 0.2%, about 0.2% to 0.4%, about 0.4% to 0.6%, about 0.6% to 0.8%, about 0.8% to 1%, about 1% to 3%, or about 3% to 5%; such as, about 0.1%, about 0.2%, about 0.3%, about 0.4%, about 0.5%, about 0.6%, about 0.7%, about 0.8%, about 0.9%, about 1%, about 2%, about 3%, about 4%, or about 5% human serum (or a serum alternative or serum replacement);
about 1 mmol to 5 mmol (such as, about 1 mmol to 2 mmol, about 2 mmol to 3 mmol, about 3 mmol to 4 mmol, or about 4 mmol to 5 mmol (e.g., about 1 mmol, about 2 mmol, about 3 mmol, about 4 mmol, or about 5 mmol)) glutamine;
about 10 ng/ml to 150 ng/ml (such as, about 10 ng/ml to 30 ng/ml, about 30 ng/ml to 50 ng/ml, about 50 ng/ml to 70 ng/ml, about 70 ng/ml to 90 ng/ml, about 90 ng/ml to 110 ng/ml, about 110 ng/ml to 130 ng/ml, or about 130 ng/ml to 150 ng/ml (e.g., about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, or about 150 ng/ml)) IL-15;
about 1 ng/ml to 50 ng/ml (such as, about 1 ng/ml to 10 ng/ml, about 10 ng/ml to 20 ng/ml, about 20 ng/ml to 30 ng/ml, about 30 ng/ml to 40 ng/ml, or about 40 ng/ml to 50 ng/ml (e.g., about 1 ng/ml, about 5 ng/ml, about 10 ng/ml, about 15 ng/ml, about 20 ng/ml, about 25 ng/ml, about 30 ng/ml, about 35 ng/ml, about 40 ng/ml, about 45 ng/ml, or about 50 ng/ml)) IFNγ;
about 1 ng/ml to 100 ng/ml (such as, about 1 ng/ml to 5 ng/ml, about 5 ng/ml to 10 ng/ml, about 10 ng/ml to 25 ng/ml, about 25 ng/ml to 50 ng/ml, about 50 ng/ml to 75 ng/ml, or about 75 ng/ml to 100 ng/ml (e.g., about 1 ng/ml, about 5 ng/ml, about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, or about 100 ng/ml)) IL-18;
about 100 ng/ml to 1000 ng/ml (such as, about 100 ng/ml to 200 ng/ml, about 200 ng/ml to 300 ng/ml, about 300 ng/ml to 400 ng/ml, about 400 ng/ml to 500 ng/ml, about 500 ng/ml to 600 ng/ml, about 600 ng/ml to 700 ng/ml, about 700 ng/ml to 800 ng/ml, about 800 ng/ml to 900 ng/ml, or about 900 ng/ml to 1000 ng/ml (e.g., about 100 ng/ml, about 200 ng/ml, about 300 ng/ml, about 400 ng/ml, about 500 ng/ml, about 600 ng/ml, about 700 ng/ml, about 800 ng/ml, about 900 ng/ml, or about 1000 ng/ml)) of an anti-CD2 antibody (e.g., RPA-2.10, TS2/18, AB75) or an antigen-binding fragment thereof; and
about 0.1 μg/ml to 10 μg/ml (such as, about 0.1 μg/ml to 0.25 μg/ml, about 0.25 μg/ml to 0.5 μg/ml, about 0.5 μg/ml to 0.75 μg/ml, about 0.75 μg/ml to 1 μg/ml, about 1 μg/ml to 2.5 μg/ml, about 2.5 μg/ml to 5 μg/ml, about 5 μg/ml to 7.5 μg/ml, or about 7.5 μg/ml to 10 μg/ml (e.g., about 0.1 μg/ml, about 0.25 μg/ml, about 0.5 μg/ml, about 0.75 μg/ml, about 1 μg/ml, about 2.5 μg/ml, about 5 μg/ml, about 7.5 μg/ml, or about 10 μg/ml about) of an anti-CD3 antibody (e.g., OKT3, UCHT1, HIT3a) or an antigen-binding fragment thereof.

In some instances, the methods described here may further use cell culture media known in the art. For example, one or more of cell culture media may be used during isolation of Vδ1 T cells from a biological sample. Several such basal culture media are available, including, but not limited to, complete media, such as ATM-V, Iscoves medium RPMI-1640 (Life Technologies), and CTS OPTMIZER T Cell Expansion SFM (GIBCO). The medium may be supplemented with other media factors, such as serum, serum proteins and selective agents, such as antibiotics. For example, in some instances, RPMI-1640 medium containing 2 mM glutamine, 10% FBS, 10 mM HEPES, pH 7.2, 1% penicillin-streptomycin, sodium pyruvate (1 mM; Life Technologies), non-essential amino acids (e.g., 100 μM Gly, Ala, Asn, Asp, Glu, Pro and Ser; 1×MEM non-essential amino acids Life Technologies), and 10 μl/L β-mercaptoethanol can be used. The cells can be cultured at 37° C. in a humidified atmosphere containing 5% CO₂ in a suitable cell culture medium.

The isolation and/or expansion method described here may comprise use of a suitable system, including, but not limited to, stirred tank fermenters, airlift fermenters, roller bottles, culture bags or dishes, and other bioreactors (e.g., hollow fiber bioreactors). The use of such systems is known in the art. Also, general methods and techniques for isolation of T cells are known in the art.

### Methods of Treatment

The γδ T cells (e.g., Vδ1 T cells) obtained by the methods described herein can be used as a medicament, for example, for adoptive T cell therapy. This may involve the transfer of the Vδ1 T cells described herein into a subject, such as, to a patient. The therapy may be autologous, where the Vδ1 T cells may be transferred back into the same subject from which they were obtained, or the therapy may be allogeneic, where the Vδ1 T cells from one subject may be transferred into a different subject. In instances involving allogeneic transfer, the Vδ1 T cells may be substantially free of UP T cells. For example, αβ T cells may be depleted from the γδ T cell population, e.g., after expansion, using any suitable means known in the art (e.g., by negative selection, e.g., using magnetic beads). A method of treatment may include: providing a biological sample (e.g., blood) obtained from a donor individual; culturing the γδ T cells from the sample as described above to produce an expanded population; and administering the expanded population of γδ T cells to a recipient individual.

As used herein, “subject” or “subject in need” can refer to an animal, of any gender (e.g., male or female), at any age (e.g., neonatal, infant, toddler, child, adolescent, adult, etc.), who is in need of a therapeutic intervention. The subject can be in need of therapeutic intervention due to a disease, such as, a cancer (e.g., a solid tumor) or an infection. The “subject” or “subject in need” can be any animal, such as a domesticated animal, a zoo animal, or a human. The “subject” or “subject in need” can be a bird or a mammal (e.g., a human or a non-human mammal). Non-human mammals may include, without limitation, rodents (e.g., mice, rats, hamsters, guinea pigs, etc.), cats, dogs, rabbits, sheep, pigs, horses, bovines, and non-human primates (e.g., chimpanzees, macaques, apes, etc.). The subject to be treated by the present methods can be a human, such as, a human patient, e.g., a human cancer patient (e.g., a human cancer patient with a solid tumor) or a virus-infected patient (e.g., a CMV-infected or HIV infected patient). In some instances, the patient has and/or is being treated for a solid tumor.

As used herein, a “solid tumor” may refer to any cancer of body tissue other than blood, bone marrow, or the lymphatic system. Solid tumors can be further divided into those of epithelial cell origin and those of non-epithelial cell origin. Examples of epithelial cell solid tumors can include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin can include sarcomas, brain tumors, and bone tumors.

Because they are normally resident in non-hematopoietic tissues, tissue-resident Vδ1 T and DN γδ T cells are also more likely to home to and be retained within tumor masses than their systemic blood-resident counterparts and adoptive transfer of these cells is likely to be more effective at targeting solid tumors and potentially other non-hematopoietic tissue-associated immunopathologies.

As γδ T cells are non-MHC restricted, they do not recognize a host into which they are transferred as foreign, which means that they are less likely to cause graft-versus-host disease (GvHD). This means that the Vδ1 T cells of the present disclosure can be used “off-the-shelf” and transferred into any recipient, e.g., for allogeneic adoptive T cell therapy. The present disclosure provides compelling evidence for the practicality and suitability for the clinical application of the γδ T (e.g., the Vδ1 T) cells obtained by the instant methods as an “off-the-shelf” immunotherapeutic reagent. These cells possess innate-like killing, have no MHC restriction and display improved homing to and/or retention within tumors than do other T cells.

A method of treatment of a subject (e.g., a human patient) with a tumor (e.g., a solid tumor) may include: providing a biological sample (e.g., blood or tissue) obtained from the subject or from a donor individual; culturing Vδ1 T cells from the biological sample as described above to produce an expanded population of Vδ1 T cells; and administering the expanded population of Vδ1 T cells to the subject with the tumor.

Pharmaceutical compositions for use in the present methods of treatment may include expanded tissue-resident Vδ1 T cells as described herein in combination with one or more pharmaceutically or physiologically acceptable carrier, diluents, or excipients. Such compositions may include buffers such as neutral buffered saline, phosphate buffered saline and the like; carbohydrates, such as, glucose, mannose, sucrose or dextrans, mannitol; proteins; polypeptides or amino acids, such as, glycine; antioxidants; chelating agents, such as, EDTA or glutathione; adjuvants (e.g., aluminum hydroxide); and preservatives. Cryopreservation solutions which may be used in the pharmaceutical compositions can include, for example, DMSO. Compositions can be formulated, e.g., for intravenous administration. The pharmaceutical composition can be substantially free of (e.g., there are no detectable levels of) any contaminant, e.g., of endotoxin or mycoplasma.

A therapeutically effective amount of expanded γδ T cells (e.g., Vδ1 T cells) obtained by the aforementioned methods can be administered in a therapeutically effective amount to a subject, e.g., for treatment of cancer (e.g., for treatment of a solid tumor). In some instances, the therapeutically effective amount of the expanded Vδ1 T cells is less than 10×10¹² cells per dose (e.g., less than 9×10¹² cells per dose, less than 8×10¹² cells per dose, less than 7×10¹² cells per dose, less than 6×10¹² cells per dose, less than 5×10¹² cells per dose, less than 4×10¹² cells per dose, less than 3×10¹² cells per dose, less than 2×10¹² cells per dose, less than 1×10¹² cells per dose, less than 9×10¹¹ cells per dose, less than 8×10¹¹ cells per dose, less than 7×10¹¹ cells per dose, less than 6×10¹¹ cells per dose, less than 5×10¹¹ cells per dose, less than 4×10¹¹ cells per dose, less than 3×10¹¹ cells per dose, less than 2×10¹¹ cells per dose, less than 1×10¹¹ cells per dose, less than 9×10¹⁰ cells per dose, less than 7.5×10¹⁰ cells per dose, less than 5×10¹⁰ cells per dose, less than 2.5×10¹⁰ cells per dose, less than 1×10¹⁰ cells per dose, less than 7.5×10⁹ cells per dose, less than 5×10⁹ cells per dose, less than 2.5×10⁹ cells per dose, less than 1×10⁹ cells per dose, less than 7.5×10⁸ cells per dose, less than 5×10⁸ cells per dose, less than 2.5×10⁸ cells per dose, less than 1×10⁸ cells per dose, less than 7.5×10⁷ cells per dose, less than 5×10⁷ cells per dose, less than 2.5×10⁷ cells per dose, less than 1×1⁰⁷ cells per dose, less than 7.5×10⁶ cells per dose, less than 5×10⁶ cells per dose, less than 2.5×10⁶ cells per dose, less than 1×10⁶ cells per dose, less than 7.5×10⁵ cells per dose, less than 5×10⁵ cells per dose, less than 2.5×10⁵ cells per dose, or less than 1×10⁵ cells per dose).

In some instances, the therapeutically effective amount of the expanded Vδ1 T cells is less than 10×10¹² cells over the course of treatment (e.g., less than 9×10¹² cells, less than 8×10¹² cells, less than 7×10¹² cells, less than 6×10¹² cells, less than 5×10¹² cells, less than 4×10¹² cells, less than 3×10¹² cells, less than 2×10¹² cells, less than 1×10¹² cells, less than 9×10¹¹ cells, less than 8×10¹¹ cells, less than 7×10¹¹ cells, less than 6×10¹ cells, less than 5×10¹¹ cells, less than 4×10¹¹ cells, less than 3×10¹¹ cells, less than 2×10¹¹ cells, less than 1×10¹¹ cells, less than 9×10¹⁰ cells, less than 7.5×10¹⁰ cells, less than 5×10¹⁰ cells, less than 2.5×10¹⁰ cells, less than 1×10¹⁰ cells, less than 7.5×10⁹ cells, less than 5×10⁹ cells, less than 2.5×10⁹ cells, less than 1×10⁹ cells, less than 7.5×10⁸ cells, less than 5×10⁸ cells, less than 2.5×10⁸ cells, less than 1×10⁸ cells, less than 7.5×10⁷ cells, less than 5×10⁷ cells, less than 2.5×10⁷ cells, less than 1×10⁷ cells, less than 7.5×10⁶ cells, less than 5×10⁶ cells, less than 2.5×10⁶ cells, less than 1×10⁶ cells, less than 7.5×10⁵ cells, less than 5×10⁵ cells, less than 2.5×10⁵ cells, or less than 1×10⁵ cells over the course of treatment).

In some instances, a dose of expanded Vδ1 T cells as described herein comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. Additionally, or in the alternative, a dose of expanded Vδ1 T cells can comprise at least about 1×10⁶, 1.1×10⁶, 2×10⁶, 3.6×10⁶, 5×10⁶, 1×10⁷, 1.8×10⁷, 2×10⁷, 5×10⁷, 1×10, 2×10⁸, or 5×10⁸ cells/kg. In some instances, a dose of expanded Vδ1 T cells comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. Additionally, or in the alternative, a dose of expanded Vδ1 T cells can comprise about 1.1×10⁶1.8×10⁷ cells/kg. In some instances, a dose of expanded Vδ1 T cells comprises about 1×10⁷, 2×10⁷, 5×10⁷, 1×10⁸, 2×10⁸, 5×10⁸, 1×10⁹, 2×10⁹, or 5×10⁹ cells. Additionally, or in the alternative, a dose of expanded Vδ1 T cells can comprise at least about 1×10⁷, 2×10⁷, 5×10⁷, 1×10⁸, 2×10⁸, 5×10⁸, 1×10⁹, 2×10⁹, or 5×10⁹ cells. In some instances, a dose of expanded Vδ1 T cells comprises up to about 1×10⁷, 2×10⁷, 5×10⁷, 1×10⁸, 2×10⁸, 5×10⁸, 1×10⁹, 2×10⁹, or 5×10⁹ cells.

In some instances, the subject is administered 10⁴ to 10⁶ expanded γδ T cells (e.g., e.g., Vδ1 T cells, such as, Gen 2 cells) per kg body weight of the subject. In some instances, the subject receives an initial administration of a population of Vδ1 T cells (e.g., an initial administration of 10⁴ to 10⁶ Vδ1 T cells per kg body weight of the subject, e.g., 10⁴ to 10⁵ Vδ1 T cells per kg body weight of the subject), and one or more (e.g., 2, 3, 4, or 5) subsequent administrations of expanded Vδ1 T cells (e.g., one or more subsequent administration of 10⁴ to 10⁶ expanded Vδ1 T cells per kg body weight of the subject, e.g., 10⁴ to 10⁵ expanded Vδ1 T cells per kg body weight of the subject). In some instances, the one or more subsequent administrations are administered less than 15 days, e.g., 14, 13, 12, 11, 10, 9, 8, 7, 6, 5, 4, 3, or 2 days after the previous administration, e.g., less than 4, 3, or 2 days after the previous administration. In some instances, the subject receives a total of about 10⁶ Vδ1 T cells per kg body weight of the subject over the course of at least three administrations of a population of Vδ1 T cells, e.g., the subject receives an initial dose of 1×10⁵Vδ1 T cells, a second administration of 3×10⁵ Vδ1 T cells, and a third administration of 6×10⁵Vδ1 T cells, and, e.g., each administration is administered less than 4, 3, or 2 days after the previous administration.

The γδ T cells (e.g., Vδ1 T cells) obtained by the instant method may also be used for CAR-T therapy. This involves the generation of engineered T cell receptors (TCRs) to re-program the T cell with a new specificity, e.g., the specificity of a monoclonal antibody. The engineered TCR may make the T cells specific for malignant cells and therefore useful for cancer immunotherapy. For example, the T cells may recognize cancer cells expressing a tumor antigen, such as a tumor associated antigen that is not expressed by normal somatic cells from the subject tissue. Thus, the CAR-modified T cells may be used for adoptive T cell therapy of, for example, cancer patients.

One or more additional therapeutic agents can also be administered to the subject. The additional therapeutic agent may be selected from the group consisting of an immunotherapeutic agent, a cytotoxic agent, a growth inhibitory agent, a radiation therapy agent, an anti-angiogenic agent, or a combination thereof. The additional therapeutic agent may be administered concurrently with, prior to, or after administration of the expanded Vδ1 T cells. The additional therapeutic agent may be an immunotherapeutic agent, which may act on a target within the subject's body (e.g., the subject's own immune system) and/or on the transferred Vδ1 T cells.

The administration of the compositions (e.g., compositions comprising the Vδ1 T cells described herein) may be carried out in any convenient manner. The compositions n may be administered to a patient transarterially, subcutaneously, intradermally, intratumorally, intranodally, intramedullary, intramuscularly, by intravenous injection, or intraperitoneally, e.g., by intradermal or subcutaneous injection. In some instances, a composition of Vδ1 T cells is directly injected into a tumor, lymph node, or site of infection.

As used herein, “treatment” (and grammatical variations thereof, such as “treat” or “treating”) may refer to clinical intervention, whether of a human or an animal (e.g., in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Also contemplated here is “treatment” as a prophylactic measure (i.e., prophylaxis). For example, a patient, subject, or individual susceptible to or at risk of the occurrence or re-occurrence of cancer may be treated as described herein. Such treatment may prevent or delay the occurrence or re-occurrence of cancer in the patient, subject, or individual. In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth can include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens. Reducing immune suppression in cancerous tumors in an individual may improve the capacity of the individual to resist cancer growth, in particular, growth of a cancer already present the subject and/or decrease the propensity for cancer growth in the individual. In some instances, expanded γδ T cells (e.g., Vδ1 T cells) are administered to delay the development of a disease or to slow the progression of a disease, e.g., to delay the development of a cancer or to slow the progression of a cancer.

As used herein, “administering” can refer to a method of giving a dosage of a therapy (e.g., an adoptive T cell therapy comprising, e.g., Vδ1 T cells) or a composition (e.g., a pharmaceutical composition, e.g., a pharmaceutical composition including Vδ1 T cells) to a patient. The compositions utilized in the methods described herein can be administered, for example, intramuscularly, intravenously, intradermally, subcutaneously, intraarterially, intraperitoneally, intracranially, intravaginally, intrarectally, intratumorally, peritoneally, subconjunctivally, mucosally, intrapericardially, intraocularly, intraorbitally, and/or intravitreally (e.g., by intravitreal injection). In some instances, the compositions described here can be administered, e.g., by eye drop, orally, topically, transdermally, by inhalation, by injection, by implantation, by infusion, by continuous infusion, by localized perfusion, by catheter, by lavage, in cremes, or in lipid compositions. The compositions utilized in the methods described herein can also be administered systemically or locally. The method of administration can vary depending on various factors (e.g., the therapeutic agent or composition being administered and the severity of the condition, disease, or disorder being treated).

A “therapeutically effective amount” can refer to an amount of a therapeutic agent to treat or prevent a disease or disorder in a subject. In the case of cancers, the therapeutically effective amount of the therapeutic agent (e.g., Vδ1 T) may reduce the number of cancer cells; reduce the primary tumor size; inhibit (e.g., slow to some extent and preferably stop) cancer cell infiltration into peripheral organs; inhibit (e.g., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., complete response (CR) and partial response (PR)), duration of response, and/or quality of life.

As used herein, “concurrently” can refer to administration of two or more therapeutic agents, where at least part of the administration overlaps in time. Accordingly, concurrent administration includes a dosing regimen when the administration of one or more agent(s) continues after discontinuing the administration of one or more other agent(s). For example, in some embodiments, Vδ1 T cells and an additional therapeutic agent may be administered concurrently.

As used herein, “pharmaceutical composition” refers to a preparation which is in such form as to permit the biological activity of one or more active ingredients contained therein to be effective, and which contains no additional components that are unacceptably toxic to a patient to which the formulation would be administered.

### EXAMPLES

The invention is further described in the following examples, which do not limit the scope of the invention described in the claims.

### Example 1. Materials and Methods

### Patient Recruitment

Patients attending St. Vincent's University Hospital (Dublin, Ireland) for partial colectomy or the Mater Misericordiae University Hospital (Dublin, Ireland) for hysterectomy were eligible for inclusion in this study. Fifteen CRC patients and nine endometrial cancer patients were recruited prospectively. All patients provided written informed consent. This study was carried out in accordance with the declaration of Helsinki and was reviewed and approved by the ethics committees of St. Vincent's University Hospital, Mater Misericordiae University Hospital and Trinity College Dublin, and Brigham and Women's Hospital.

Isolation of Mononuclear Cells from Resection Material

Surgical specimens were stored in RPMI+10% FCS for transport to the laboratory. Tumors and healthy tissue were finely minced using a dissection scissors and placed in cryostor CS10 reagent for cold storage at −80° C. Sample were later thawed at 37° C., cryostor was rinsed from the tissue using warm RPMI. Tissue samples were further mechanically dissociated and placed in an enzymatic digestion mixture (Collagenase type IV 0.05%, DNase in RPMI) to produce a single cell solution. Digestion solution was incubated at 37° C. for 45 min agitated at 180 rpm in a shaking incubator. Digested tissue was strained through a 100 m filter, followed by a 70 m filter. RBC lysis was performed using ACK lysis solution for 5 min at room temperature.

Isolation of PBMCs from Healthy Donors

PBMCs were isolated from anonymized healthy blood donors attending the Kraft blood donor center. PBMCs were separated by density gradient centrifugation using FICOLL-PAQUE™ PLUS (GE Healthcare) and residual red blood cells were removed by adding red cell lysis solution.

### Performance of Flow Cytometry Analysis

Single cell suspensions were stained in FACS buffer for 30 min in the dark with fluorescently labeled monoclonal antibodies. A full list of antibodies used can be found in Table 1. Dead cell exclusion was performed using fixable viability stain ZOMBIE AQUA (Biolegend). Mitochondrial mass was assessed using MITOTRACKER GREEN (Thermo Fischer); mitochondrial membrane potential was assessed using TMRM. MTG and TMRM were diluted in RPMI to appropriate concentrations. Cells were resuspended in this MTG/TMRM solution and incubated at 37° C. for 20 min. Intracellular staining was performed using CYTOFAST FIX/PERM kit (Biolegend). Cells were fixed for 20 min at room temperature, permeabilized and intracellular antibodies incubated for 30 min. Flow cytometric analysis was performed using an LSR FORTESSA (BD Biosciences) and data were analyzed using FLOWJO (Version 10.8.0, Tree Star, Ashland, OR, USA).

TABLE 1
Antibodies to identify Vδ1 and their subsets

| Target | Clone | Supplier |
|---|---|---|
| CD45 | H130 | BioLegend |
| CD3 | SK7 | BioLegend |
| CD8 | RPA-T8 | BioLegend |
| CD56 | 5.1H11 | BioLegend |
| VD1 | TS8.2 | Invitrogen |
| VD2 | B6 | BioLegend |
| TIGIT | 741182 | BD |
| LAG3 | 11C3C65 | BioLegend |
| TIM3 | F38-2F2 | BioLegend |
| PD1 | EH12.2H7 | BioLegend |
| TNFa | MAb11 | Invitrogen |

### Preparing Single Cell RNAseq Samples

Single-cell RNA-seq was performed on single cell suspensions of sorted innate T cells and NK cells from 7 human colorectal, 2 endometrial tumors, and 7 adjacent normal tissue using the 10×Genomics platform. Human colorectal, endometrial tumors and adjacent normal tissues were minced, stored in CRYOSTOR solution and frozen at −20° C. Samples were then thawed and digested into single cell suspensions before FACS sorting to enrich for innate T cells and NK cells. Cell suspensions were barcoded (10× CHROMIUM SINGLE CELL PLATFORM CONTROLLER, 10× Genomics) to generate single cell Gel Beads-in-emulsion (GEMS) and GEMs were processed to generate UMI-based libraries according to the Chromium Single Cell 3′ Library protocol. All libraries were sequenced using an ILLUMINA NEXTSEQ 500 sequencer resulting in eight datasets (two each for tumor as well as matched normal). Raw BCL files were demultiplexed using CELL RANGER V3.0.2 MKFASTQ to generate fastq files with default parameter. Fastq files were aligned to the human genome version 38 (GRCH38) and feature reads were quantified simultaneously using CELL RANGER count for feature barcoding. The resulting filtered feature-barcode UMI count matrices containing quantification of gene expression and hashtag antibody binding were then utilized for downstream analysis. Use of the 10× Genomics platform and initial sample quality control was performed by the BWH Center for Cellular Profiling.

Preprocessing of scRNAseq Data

scRNAseq data was loaded into SEURAT (v4.0.6, R version) from feature-barcode UMI count matrices. Datasets from all 5 colorectal and 2 endometrial data sets were merged using the MERGESEURAT function. Then, data from low-quality cells were subsetted, cells with no hashtag reads and cells with high mitochondrial reads were removed, cell cycle regression was performed, and any remaining doublets were removed using SCRUBLET (v 0.0.2, R version). After observing the overall gene expression distributions of every sample, we excluded cells with less than 200 genes as well as low-quality genes defined as genes expressed in less than 3 cells. Cells expressing less than 1% minimum or more than 25% maximum of mitochondrial genes as a % of total gene counts were considered to represent empty droplets or apoptotic/dead cells and were removed from the analysis. Cells were also filtered based on total UMI counts and total gene counts on a per sample basis to remove empty droplets, poor quality cells and doublets, with a minimum cutoff of at least 1000 genes per cell across all samples. Doublets were identified in each 10× sample individually using SCRUBLET, setting the expected doublet rate to 0.03. Cells were excluded when they had a score higher than 0.05 for samples. UMI counts were normalized, and log transformed using regularized negative binomial regression using SCTRANSFORM (v0.3.271, R version). Where indicated, cycle regression was performed by first normalizing UMI counts using SCTRANSFORM, then performing cell cycle scoring using the CELLCYCLESCORING function and cell cycle gene lists provided with the SEURAT package, and then re-normalizing raw RNA count data with SCTRANSFORM and regression of computed cell cycle scores applied.

Downstream Analysis of scRNAseq Data

The downstream analyses included, normalization, scaling, clustering of cells and identifying cluster marker genes. Our scRNAseq datasets were converted to h5ad files compatible with python and were loaded into software package SCANPY (v 1.8.1, python). Highly variable genes were found within the dataset and then principal component analysis (PCA) dimensionality reduction of n=50 pcs and was performed with the highly variable genes as input. The PCs were then used to calculate nearest neighbor graphs and UNIFORM MANIFOLD APPROXIMATION AND PROJECTION (UMAP) for each dataset. When necessary for collective analysis of cells from different batches, the HARMONYPY (v1.0 package, Python version) was used with default settings to remove batch effects, and batch-corrected harmony embeddings were used for UMAP. UMAP was performed using a minimum distance of 0.3 and a spreading factor of 1.

Clustering into subgroups was performed based on these gene sets. Using the LEIDEN algorithm (leidenalg v 0.8.9, Python version), and the calculated nearest neighbor graphs, we identified our main clusters. In some cases, over clustering was performed and clusters were manually collapsed, and/or the first two dimensions of the UMAP reduction were used as input for graph-based clustering instead of PCA or harmony embeddings. After separation between cell types, we performed differential gene expression. Rank_genes_groups function in SCANPY was used to find markers of each LEIDEN cluster using the Wilcoxon rank sum test method on log-normalized RNA counts. Heatmap, violin plots, linear regression plots, and UMAP visualizations shown in this paper were prepared using SCANPY.

Integrated Analysis of Public scRNAseq Data

Integrated analysis of public scRNAseq data was completed in a similar methodology as listed above. We were granted early access to the Flagship Human Colon Cancer Atlas as raw count matrix. In addition to the cell annotations provided in the original meta-data, we annotated the cells from public datasets by comparing the differential markers between clusters against cell markers from our enriched sequencing dataset.

### TCGA Gene Expression and Survival Analysis

Data was downloaded using TCGAbiolinks for projects TCGA-BRCA, TCGA-COAD, TCGA-LUAD, TCGA-OV and TCGA-UCEC. Clinical survival data was downloaded from supplementary data provided in Liu et al., 2018). Survival analysis used RPARTSURVIVALCLASSIFIER method based on the rpart package (Therneau, T., Atkinson, B., & Ripley, B. (2013). Rpart: Recursive Partitioning. R Package Version 4.1-3, available at CRAN.R-project.org/package=rpart) which allows stratification of the patient cohort based on gene expression and survival information. We also ran a conventional median gene expression stratification method. Survival analysis used the survival package (Therneau T (2021). A Package for Survival Analysis in R. R package version 3.2-13, available at CRAN.R-project.org/package=survival). We used the survminer package (available at github.com/kassambara/survminer) for visualization. Colorectal and endometrial tumor data sets were downloaded from the TCGA and GTEX databases through the Genomic Data Commons Data Portal using TCGAbiolinks. Gene expression data was plotted using ggplot2. Patient samples were filtered by MSI status, KRAS mutation status and consensus molecular classification was used to stratify colon cancer patients using CMSCaller.

### Expansion of Vδ1 T Cells

Vδ1 T cells were isolated from healthy blood donors attending the Kraft Blood donor center, BWH. PBMCs were stained with anti-Vδ1 PE (Miltenyi) antibody for 30 min at 4° C. Anti-PE antibody was then added and incubated for 15 min at 4° C. Cell separation was performed using an AUTOMACS Pro Separator. Isolated Vδ1 T cells were then expanded (Almeida et al., Clin. Cancer Res. 22, 5795-5804 (2016)). Briefly, Vδ1 T cells were expanded for 2 weeks in animal-free T cell media (OPTIMIZER-CTS) with 5% human serum, 2 mmol glutamine in the presence of IL-1β (15 ng/ml), IL-4 (100 ng/ml), IL-21 (7 ng/ml), IFNγ (70 ng/ml), anti-CD3 (70 ng/ml). After 2 weeks media was replaced with T cell media containing IL-15 (70 ng/ml), IFNγ (30 ng/ml) and anti-CD3 (1 μg/ml), for a further 7 days.

### Wound Healing Scratch Assay

Human colon cell lines SW480 and HCT116 were passaged twice before use. 24-well plates were coated with 200,000 cells and allowed to adhere and form a monolayer overnight. The following day a scratch was performed using a sterile p200 pipette tip forming a cross in the same central location in each well. Cells were then washed in warm PBS to remove cellular debris. DMEM was then added to each well, with or without Vδ1 T cells at a concentration of 1×10⁵/ml. Wells were imaged immediately after performance of the scratch, 6 hrs and 24 hrs later using an Olympus CKX41 light microscope. In additional experiments, SW480 cells were treated with cetuximab (1 μg/ml) for 1 hr after scratching before Vδ1 T cells were added. Image analysis was performed using IMAGEJ and the MRI wound healing tool (available at dev.mri.cnrs.fr/attachments/download/1992/MRI_Wound_Healing_Tool.ijm).

The scratch size was measured in pixels and percentage change over time was calculated. After measurement of the scratch, suspension cells (Vδ1 T cells) were aspirated and used for flow cytometry. Supernatants were collected and used to measure AREG secretion by ELISA.

### Production of Metabolically Fit Vδ1 T Cells

Vδ1 T cells were expanded for 7 days in the presence of anti-CD3 (1 g/ml)±IL-10, IL-4, IL-21, IFNγ, IL-15. After 7 days they were stimulated with PMA for 4 hrs and assessed for AREG and IFNγ production. Based on these results anti-CD3+IL-15 was retained for future use. This base was then tested in combination with innate and adaptive cytokine cocktails containing IL-12 (50 ng/ml), IL-18 (50 ng/ml), IL-2 (1000 U/ml), anti-CD2 (1 μg/ml) for 3 weeks. Based on these preliminary experiments which examined cell numbers, Vδ1 purity, and immune checkpoint expression, the GEN 2 protocol was established combining IL-15 (70 ng/ml), IL-18 (50 ng/ml), anti-CD2 (500 ng/ml) & anti-CD3 (1 g/ml).

### Performance of Bulk RNAseq and Data Analysis

Unexpanded, Gen 1 or Gen 2 cells were cultured for 4 hours with or without PMA stimulation. Cells were then placed in lysis solution and prepared for RNA isolation using RNEASY PLUS Mini kit (Qiagen). RNA samples were submitted to the Molecular Biology Core Facility at Dana-Farber Cancer Institute. Total RNA was enriched for polyadenylated transcripts using oligo-DT coated beads from Kapa mRNA Hyper Prep kit (Roche). RNA quality was assayed using an Agilent Bioanalyzer, with RIN/RQN values >7 used. Sequencing was performed on a ILLUMINA NEXTSEQ500 platform. Gene alignment was performed using STAR V2.7. 10a. VIPER visualization was used to assess sample quality. Differential gene expression was performed using DESeq2. Data visualization was performed using GGPLOT2, PHEATMAP, ENHANCEDVOLCANO.

### Cytotoxicity Assay

Target cells (K562) were labelled with cell trace violet (CTV). Expanded Vδ1 T cells were incubated with target cells at a various ratio (0.25:1, 0.5:1, 1:1, 5:1, 10:1) for 4 hours. Cytotoxicity was assessed by propidium iodide staining (PI). CTV+ cells were gated on and PI+ cells measured as a percentage of target cells.

### Seahorse Metabolic Flux Analysis

Real-time analysis of oxygen consumption rates (OCR) and extracellular-acidification rates (ECAR) of GEN 2 and Gen 1 cells sorted were assessed using the XFP EXTRACELLULAR FLUX (Seahorse Bioscience). 1×10⁵ Cells were added to a Seahorse Cell Culture Microplate (Agilent), coated with CELL-TAK (Corning) to ensure adherence, and sequential measurements of ECAR and OCR were performed in XF RPMI Seahorse medium supplemented with glucose (10 mM), glutamine (2 mM), and sodium pyruvate (1 mM) following the addition of Oligomycin A (2 μM), FCCP (2 μM), rotenone (1 μM) plus antimycin A (4 μM). Basal glycolysis, glycolytic capacity, basal mitochondrial respiration, and maximal mitochondrial respiration were calculated.

### Patient Derived Xenograft Production

A patient CRC tumor was obtained from the UMass Chan Medical School Cancer Avatar Institute (IRB ID: H00004721) and passaged in NSG mice to deplete the human leukocytes present within the tumor microenvironment (Maykel et al., Dig. Dis. Sci. 59, 1169-79 (2014)). The patient derived xenograft CRC was then processed into ~2×2×2 mm³ pieces and tumor fragments were transplanted subcutaneously to the right flank of NSG-Tg(Hu-IL15) mice. Tumor size was measured by caliper 2 to 3 times a week and volume (mm³) were calculated by (length×width)²/2.

### Mice

Male NOD-scidIL2rg^{null} (NSG) mice and NSG-Tg(HuIL15) mice were obtained from The Jackson Laboratory (Bar Harbor, ME). NSG-Tg(HuIL15) constitutively express human IL-15, with levels in serum detectable at 15 μg/ml. All animals were housed in a specific pathogen-free facility, in microisolator cages, and given autoclaved food and maintained on sulfamethoxazole-trimethoprim medicated water (Goldline Laboratories, FL) and acidified autoclaved water on alternating weeks. All animal use was in accordance with the guidelines of the Animal Care and Use Committee of the University of Massachusetts Chan Medical School and conformed to the recommendations in the Guide for the Care and Use of Laboratory Animals (Institute of Laboratory Animal Resources, National Research Council, National Academy of Sciences, 1996).

### Example 2. Heterogeneity of Gamma Delta (γδ) T Cell Populations Linked to Poor Outcomes in Colorectal Cancer

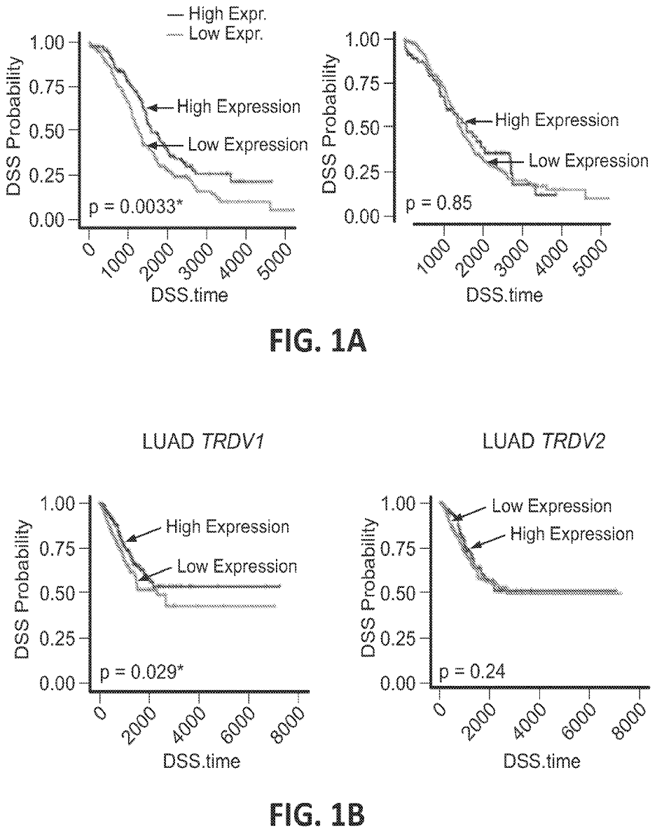

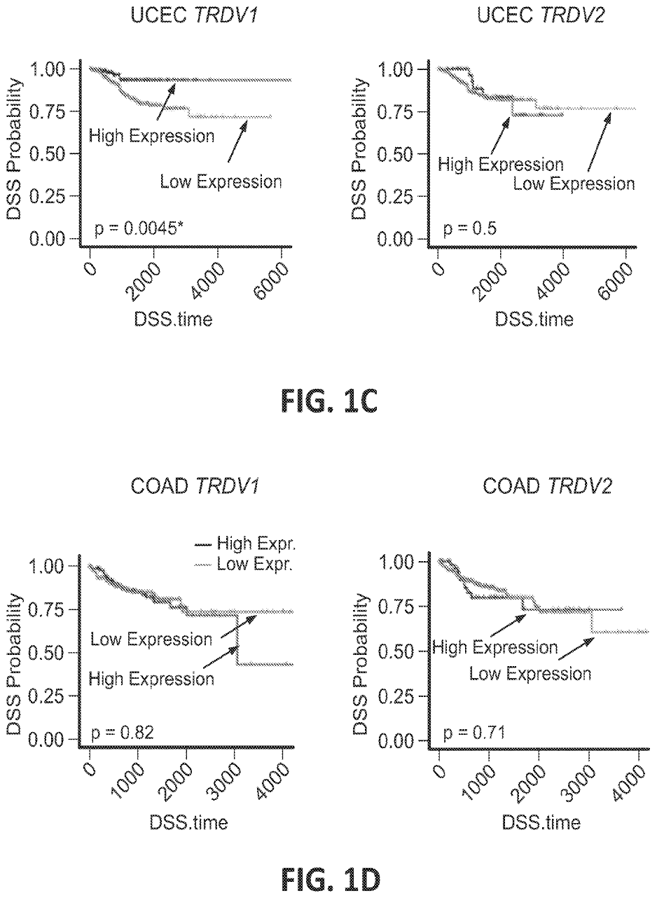

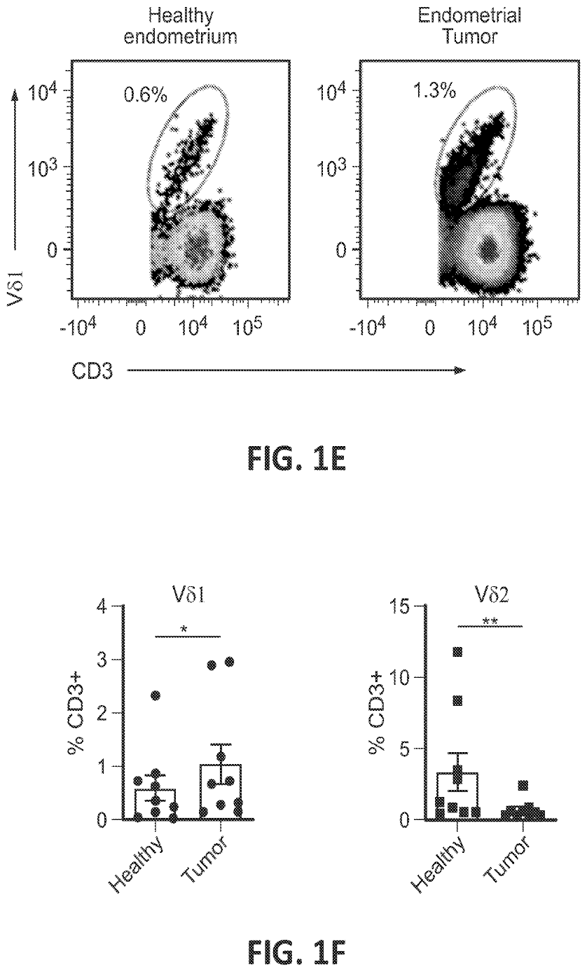

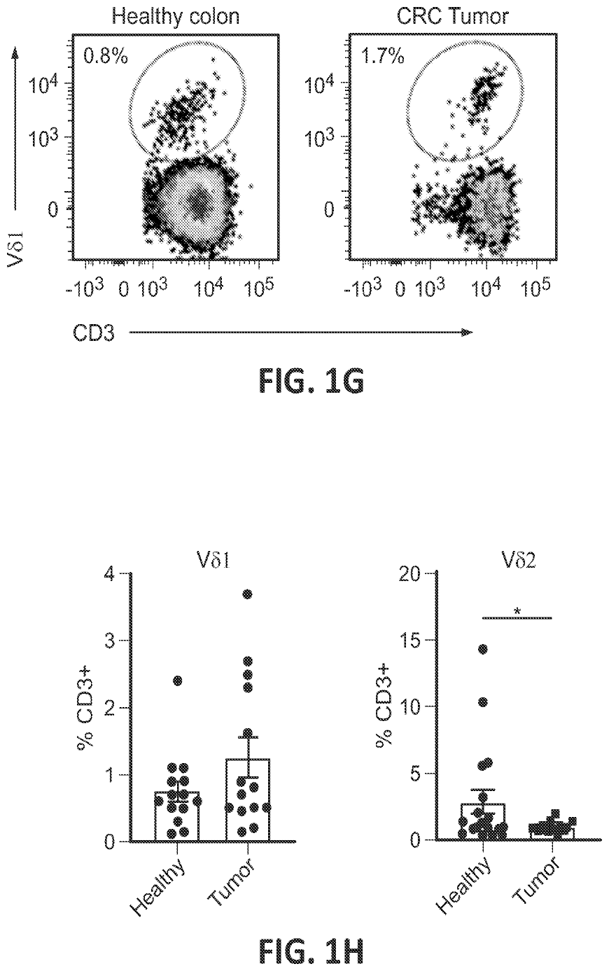

The prognostic value of γδ T cells presence in solid tumors is positive with some notable exceptions. We therefore investigated the biology of γδ T cells in cancer types that were associated with different prognoses with respect to γδ T cell enrichment. We used the expression of TRDV1 and TRDV2 to stratify patients into high or low Vδ1 and Vd2 enrichment respectively. In high grade serous ovarian cancer, lung adenocarcinoma (LUAD), and uterine corpus endometrial (UCEC) cancers, high levels of TRDV1 were associated with improved disease specific survival (FIGS. 1A-IC). In contrast, in CRC (COAD), high levels of TRDV1 were not associated with a positive prognosis (FIG. 1D). In all four tumor types, TRDV2 (Vδ2) expression showed no correlation with survival (FIGS. 1A-1D). Analysis of tumors by flow cytometry showed a significant reduction in the proportion of Vδ2 cells, but Vδ1 T cells were enriched in endometrial tumors and unchanged in CRC (FIGS. 1E-1H).

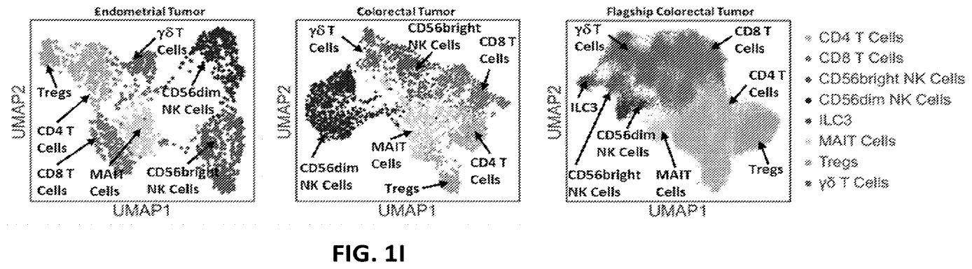

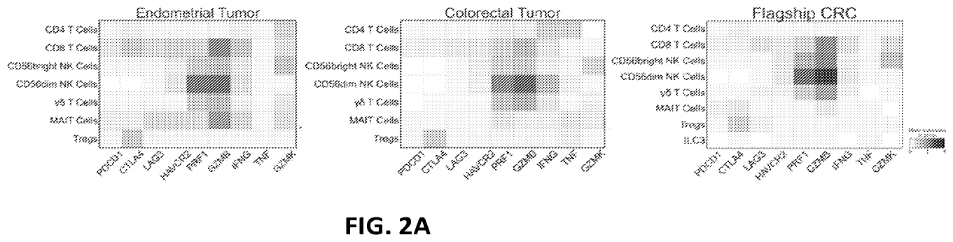

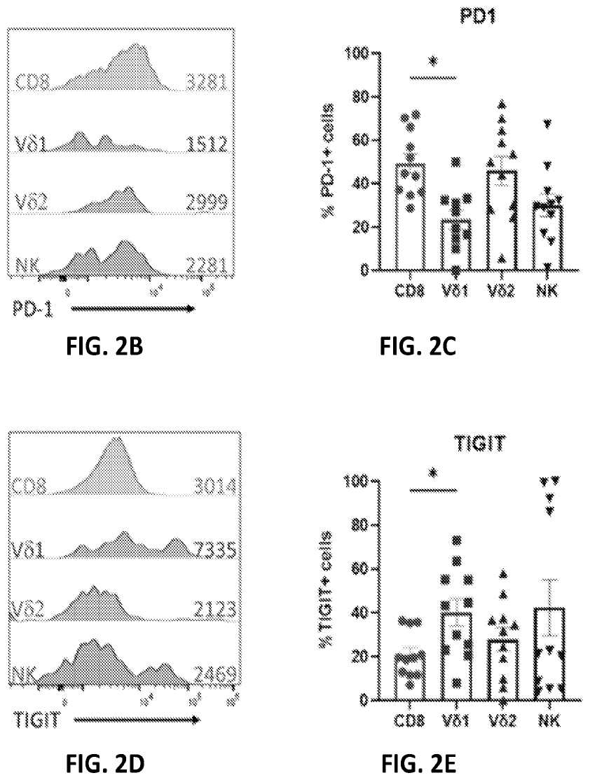

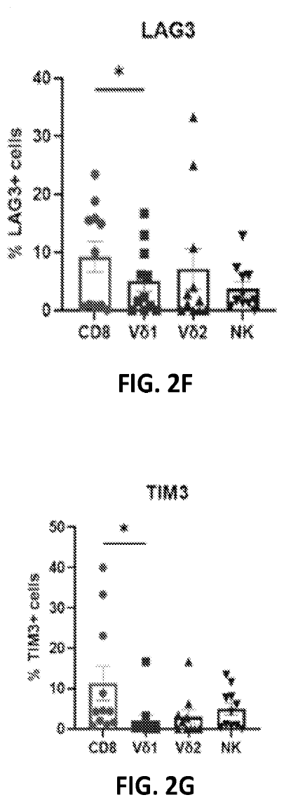

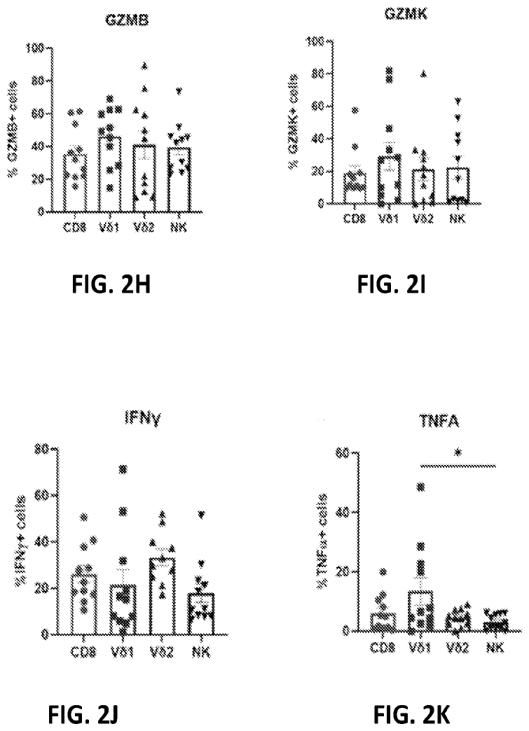

As Vδ1 cells—but not Vδ2 cells—were enriched in both tumor types, and Vδ1 level—but not Vδ2 level—correlated with patient survival, we focused our study on the Vδ1 subset of human γδ T cells. We hypothesized that the phenotype of tumor infiltrating Vδ1 cells may contribute to the differing disease outcomes associated with Vδ1 enrichment in endometrial versus colorectal cancer. To investigate this, we employed a single cell RNAseq based approach. First, we enriched for innate T cells (γδ and MAIT cells) and sequenced similar proportions of γδ T cells, MAIT cells, NK cells, Tregs and adaptive CD4 & CD8 T cells from both tumor types. Clusters were identified based on differential gene expression and expression of lineage specific genes. Gene expression correlation shows that γδ T cells are more closely associate with innate NK cells and ILCs than adaptive CD8 and CD4 T cells, in endometrial cancers and CRC (FIG. 1I). We noted that γδ T cells differ from conventional T cells in their effector, immune checkpoint and activatory (or activation) receptor expression patterns, and were more similar to NK cells than conventional T cells in their top gene expression. We also accessed a large publicly available CRC ‘Flagship’ dataset of over 60 patients to confirm our findings (FIG. 2A).

### Example 3. γδ T Cells in Tumors have Low Checkpoint Expression and High Cytotoxic Markers

Little is known about checkpoint expression on human innate T cells. We analyzed immune checkpoint, effector molecule and cytotoxicity receptor expression using scRNAseq data sets (FIG. 2A). In both endometrial cancer and CRC, γδ T cells expressed low levels of PD1 (PDCD1), CTLA4, TIM3 (HAVCR2) and LAG3, similar to NK cells, and lower than conventional T cells (FIG. 2A). In addition, they maintained high levels of cytotoxic effector molecules, granzyme B (GZMB), perforin (PRF1), and interferon gamma (IFNγ), similar to CD8 T cells and NK cells (FIG. 2A). Therefore, γδ T cells have a cytotoxic phenotype without typical inhibitory markers in 2 human cancer types. We confirmed these findings in an independent cohort of CRC patients by flow cytometry. Vδ1 T cells showed the lowest levels of expression for PD1 (FIGS. 2B-2C). However, Vδ1 T cells expressed high levels of TIGIT (FIG. 2D-2E), an inhibitory receptor previously described on NK cells. LAG3 and TIM3 were significantly lower in Vδ1 T cells compared to CD8 T cells (FIGS. 2F-2G). Furthermore, Vδ1 T cells expressed similar, and in some cases higher levels of GZMB, GZMK, IFNγ and TNFα compared to other cytotoxic lymphocyte populations (FIGS. 2H-2K). Thus, γδ T cells are a tumor-enriched cytotoxic T cell subset with potentially less inhibition than other anti-tumor populations (CD8, CD4, MAIT cells).

### Example 4. γδ T Cells are Heterogeneous in Colon Cancer but not Endometrial Cancer

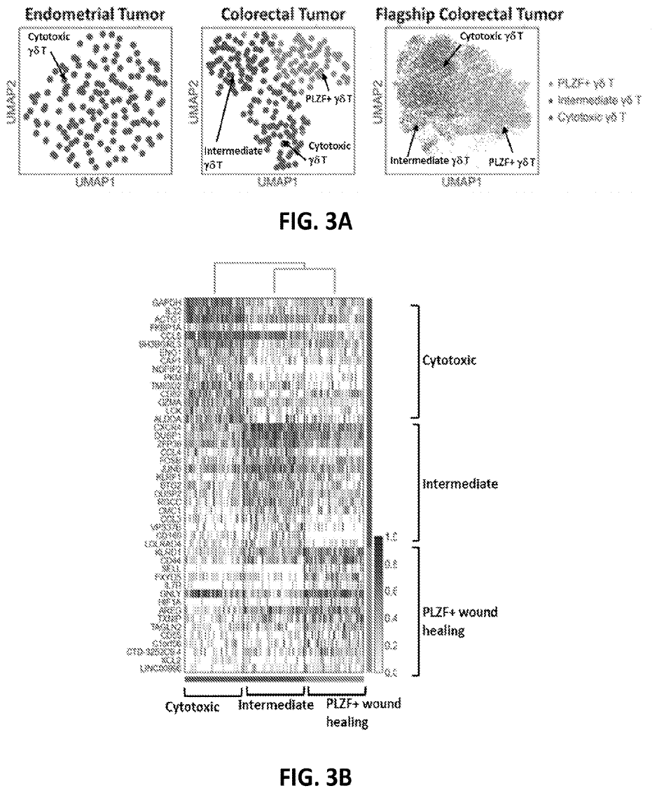

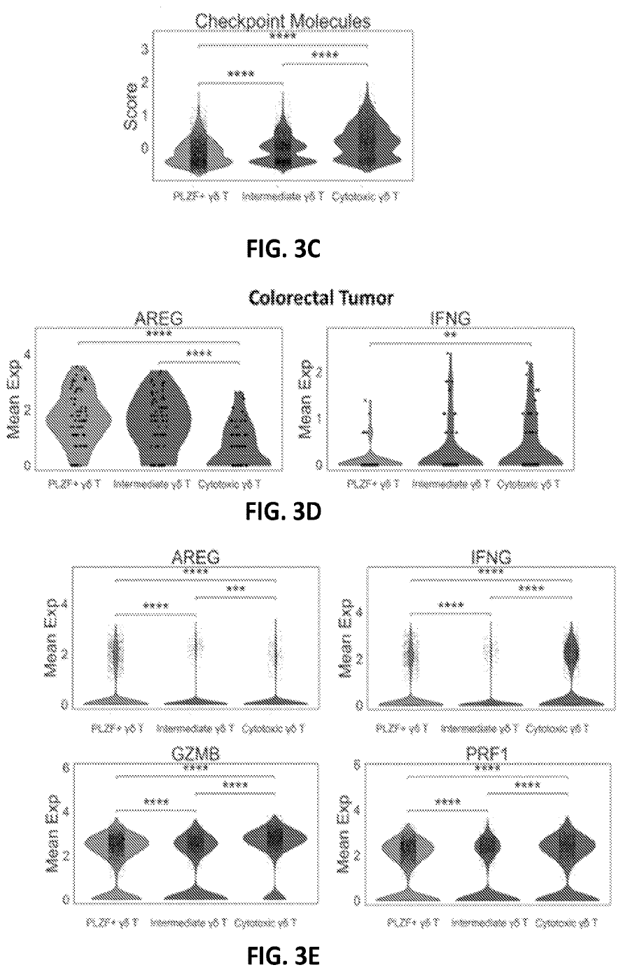

We next focused on γδ T cell clusters in endometrial cancer and CRC. Endometrial γδ T cells were homogenous, consisting of one cluster with a cytotoxic phenotype (FIG. 3A). In CRC, 78 T cells were more heterogeneous, segregating into three distinct clusters. Further analysis of the CRC γδ T cell clusters identified significant differences in their functional phenotypes, with a cytotoxic cluster (PRF1, GZMA, CCL5, ENO1, PKM, GNLY), a PLZF+ wound healing cluster (ZBTB16, AREG, TAGLN2, CD44) and an intermediate cluster expressing genes associated with tissue residence (KRLB1, CXCR4, FOSB, JUNB) but also effector molecules (FIG. 3B). A Z-score of combined immune checkpoint expression showed that it was mainly the cytotoxic cluster which expressed checkpoints, suggesting that the PLZF+ and intermediate clusters were not exhausted but rather a functionally distinct subset (FIG. 3C). Using our CRC dataset and the Flagship CRC dataset, we found that the PLZF+ cluster expressed the lowest level of effector genes including IFNγ, GZMB and PRF1, while the cytotoxic cluster showed the highest expression of these genes (FIGS. 3D-3E). AREG was significantly enriched in the PLZF+ cluster. The intermediate cluster expressed AREG but also effector molecules such as IFNγ, GZMB and PRF1, suggesting a transitionary population. Low/no AREG expression was detected in endometrial tumor infiltrating γδ T cells, which displayed a homogenous cytotoxic phenotype.

We next confirmed AREG protein production by flow cytometry. Examining tumor infiltrating γδ T cells from CRC biopsies, we found that AREG was produced by Vδ1 and Vδ3 subsets, which are typically tissue resident, while Vδ2 cells were mainly IFNγ+(FIGS. 3F-3I). However, a large proportion of Vδ1 cells also produced IFNγ, with some also producing both AREG+ and IFNγ+ cells, possibly representing the ‘intermediate’ cluster. Thus, Vδ1 T cells are heterogenous in CRC but not endometrial cancer and consist of populations that can produce IFNγ or AREG.

### Example 5. AREG Expression is Increased in CRC Tumors and Associated with Mismatch Repair Proficient Tumors

Using TCGA and GTEX data sets, we determined that AREG expression is significantly increased in CRC tumors, but not endometrial tumors compared to healthy tissue (FIGS. 4A-4B). Furthermore, AREG expression is associated with a molecular subtype of CRC. AREG is enriched in microsatellite stable (MSS) compared to microsatellite instability (MSI) tumors (FIG. 4C). Further, using consensus molecular classifications (CMS) of CRC, we determined that AREG was associated with CMS 2, or a wound healing phenotype, commonly seen in CRC (FIG. 4D).

In our datasets and the Flagship dataset, scRNAseq was also performed on matched normal adjacent endometrial and colon tissue from cancer patients. In both normal endometrium, normal colon and CRC tumors, AREG gene expression is associated with innate cells, γδ T cells, NK cells and ILC3s (FIGS. 4E-4H). In colon cancer, we identified non-lymphoid cells which also produce AREG, including dendritic cells and mast cells. Interestingly, γδ T cells were the only population that increased AREG gene expression in CRC tumors compared to normal colon (FIG. 4I). AREG protein expression was also highest in Vδ1 T cells in CRC (FIG. 4J). In normal colon, Tregs produce AREG but produce significantly less AREG in CRC (FIG. 4J). IFNγ expression was seen in many innate and adaptive cell types and was significantly increased in 78 T cells in CRC (FIGS. 4H and 4K).

As both AREG and IFNγ were increased in γδ T cells in CRC tumors, we asked if this was due, in part, to different tumor subtypes. Next, we analyzed the flagship CRC dataset separated into normal colon, and DNA mismatch repair proficient (MMRp) and deficient (MMRd) tumors (analogous to MSS and MSI respectively). MMRp and MMRd are considered immunologically “cold” and “hot” respectively. In this dataset, there were a greater number of γδ T cells from MMRd tumors compared to MMRp but were overall at a similar percentage of total lymphocytes across patients. We found striking differences in the γδ T cell phenotype between tissue and tumor type. Healthy colon is populated mainly by γδ T cells with the intermediate phenotype (FIGS. 4L-4M), potentially poised to respond to a range of functional requirements. Firstly, in both CRC tumor types, there was a loss of the ‘normal’ intermediate γδ T population typically found in normal colon. In MMRp CRC tumors, there was a striking skew towards PLZF+γδ T cells, and in MMRd tumors there was the emergence of cytotoxic γδ T cells but also PLZF+γδ T cells (FIGS. 4L-4M). This correlates with the association of cytotoxic lymphocytes with MMRd tumors, as previously described for CD8 T cells (Llosa et al., Cancer Discov. 5, 43-51 (2015); Boland et al., Gastroenterology 138, 2073-2087 (2010); Anitei et al., Clin. Cancer Res. 20, 1891-9 (2014)). Furthermore, the increased proportion of AREG expressing cells in MMRp tumors may contribute to the wound healing phenotype of these tumors and increased levels of AREG in whole tumor samples (FIGS. 4C-4D), since they were the only population to increase AREG expression in the tumor. AREG expression was seen in the PLZF+ and intermediate clusters, which are most prominent in the MMRp tumors (FIG. 4N). IFNγ expression is highest in MMRd tumors and expressed in the cytotoxic cluster (FIG. 4O). AREG expression was seen at similar levels across all patients in each molecular subtype.

### Example 6. AREG Expression is Associated with NKp80+Vδ1 Cells and Induces Proliferation of CRC Cancer Cells

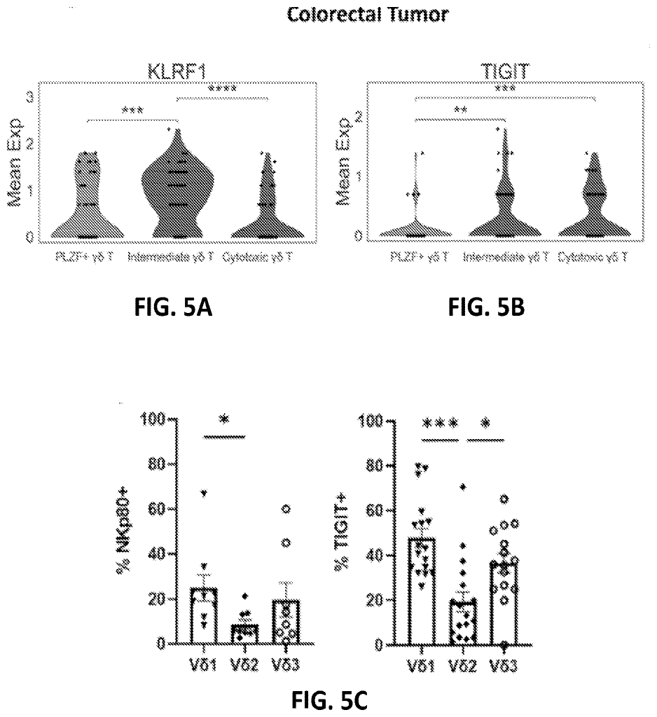

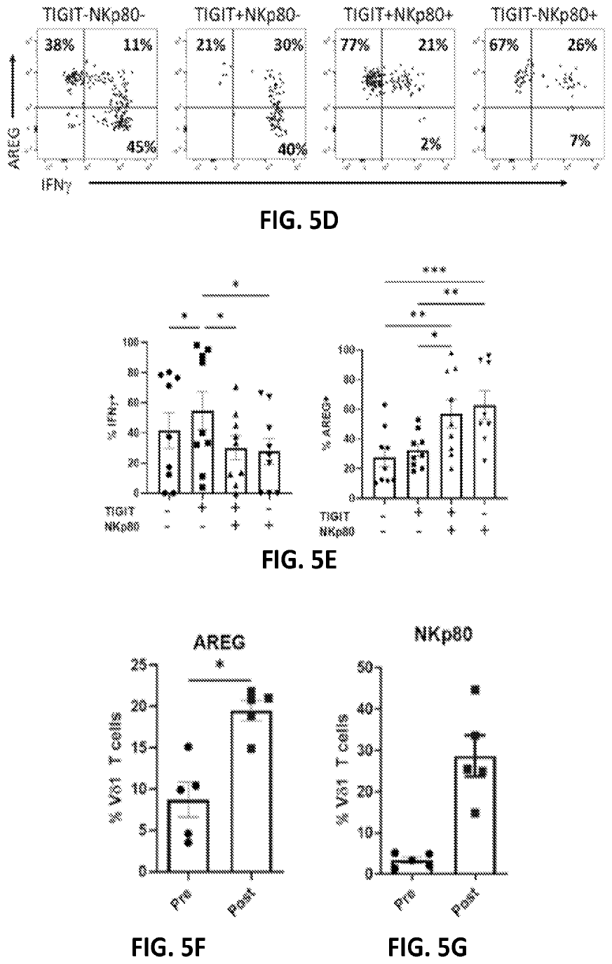

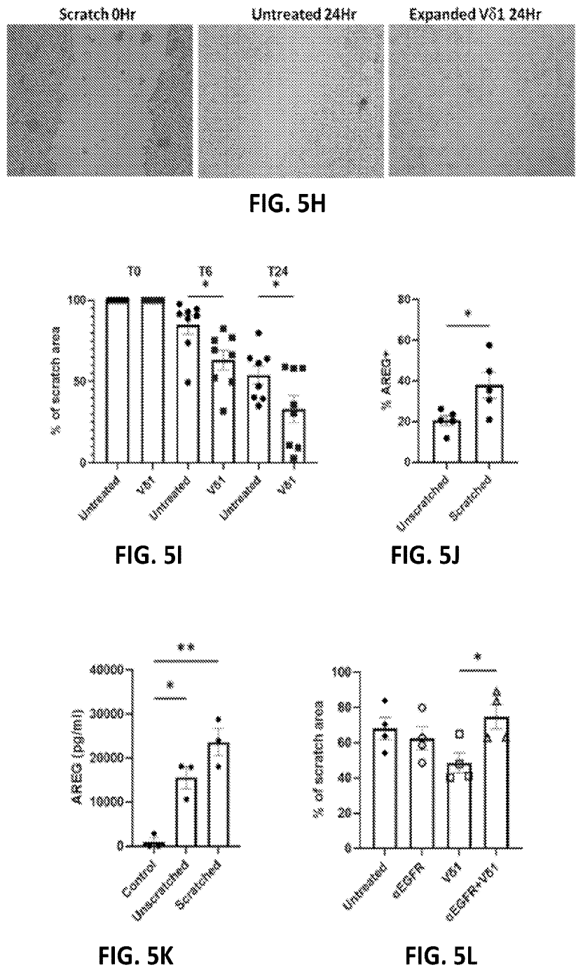

We next aimed to identify surface receptors associated with wound healing and cytotoxic γδ T cell phenotypes, so as to identify these populations more easily in future cohorts. We screened a number of cytotoxicity receptors and immune checkpoints to identify immune signatures associated with AREG or IFNγ production in Vδ1 T cells. KLRF1 (NKp80) was strongly associated with AREG expression in scRNAseq data sets, while TIGIT was associated with a cytotoxic phenotype (FIGS. 5A-5B). Both NKp80 and TIGIT were most highly expressed on Vδ1 T cells compared to Vδ2 (FIG. 5C). TIGIT+ cells had the highest levels of IFNγ production and the lowest levels of AREG production in CRC tumors. Inversely, NKp80+ cells had the highest levels of AREG production and the lowest level of IFNγ (FIG. 4D-4E).

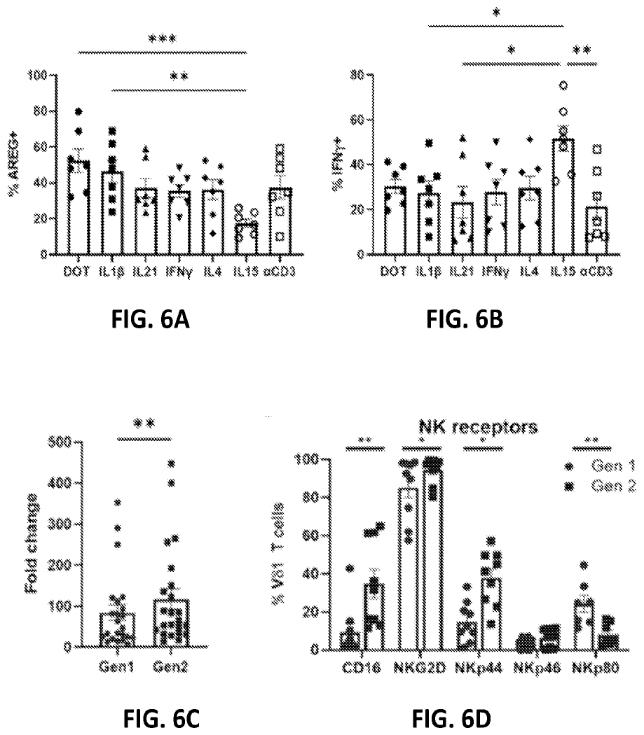

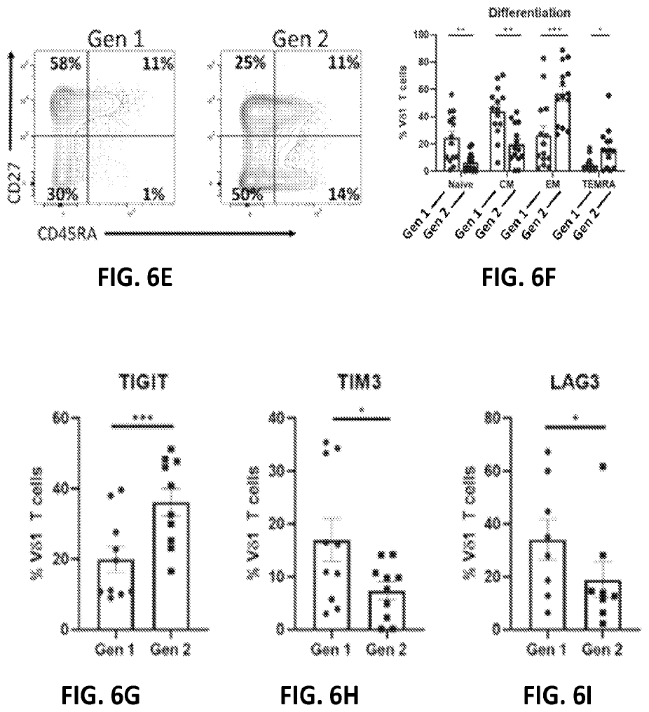

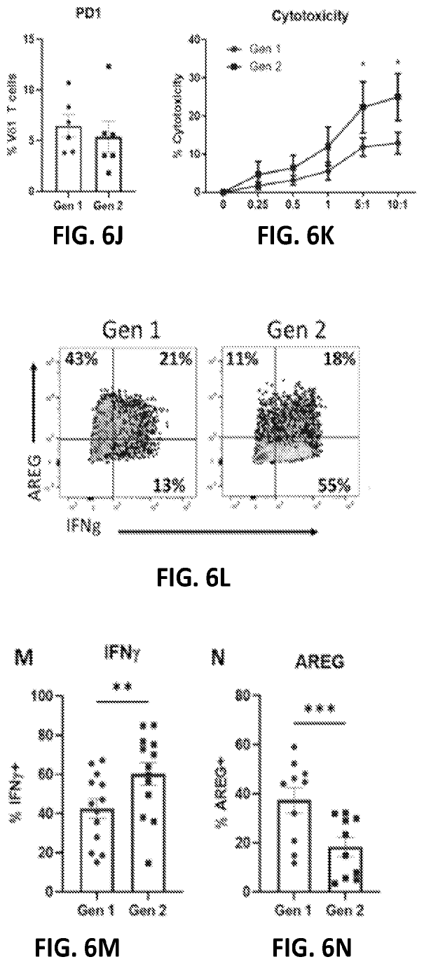

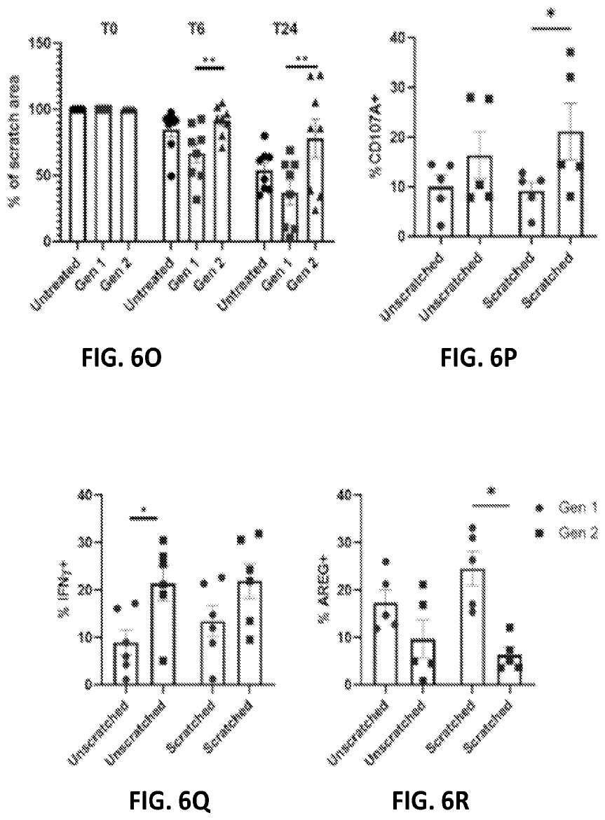

We next expanded Vδ1 T cells from healthy blood using a published protocol (Almeida et al., Clin. Cancer Res. 22, 5795-5804 (2016)). We found that AREG production increased with expansion and AREG producing Vδ1 expressed NKp80 but not TIGIT (FIGS. 5F-5G). Next, we asked if Vδ1 cells producing AREG had a wound healing function, using a scratch assay. Two human CRC tumor cell lines, SW480 and HCT116 were plated, scratched and co-cultured with the expanded Vδ1 cells. This process mimics the formation of a ‘wound’ and the migration of epithelial cells to close the wound. We found that expanded Vδ1 T cells closed the scratch area and increased tumor cell growth in both SW480 and HCT116 tumor cells (FIGS. 5H-5I). This increase in wound closure was accompanied by increased production and secretion of AREG by Vδ1 cells (FIGS. 5J-5K). Tumor migration induced by AREG was blocked by pre-treating tumor cells with cetuximab, which blocks the AREG receptor EGFR (FIG. 5L). To explore this we began with the published Vδ1 expansion protocol, which from here is termed 1^{st} generation (Gen 1), which includes Vδ1 cultured with IL-1β, IL-21, IFNγ, IL-4 and IL-15 (Almeida et al., C/in. Cancer Res. 22, 5795-5804 (2016)). We investigated if a specific cytokine component of the expansion protocol promoted the AREG phenotype, by culturing Vδ1 T cells with anti-CD3+/− each individual cytokine from the cocktail. We found that IL-1β produced the highest levels of AREG of any cytokine alone (FIG. 6A). Furthermore, when we blocked IL1β in vitro during a scratch assay, wound closure was prevented at levels comparable to cetuximab.

Thus, a significant proportion of Vδ1 expanded from healthy blood could produce AREG and had a pro-tumor, wound healing function in vitro. This may preclude Vδ1 as an effective cell therapy for some solid tumors.

### Example 7. AREG Production can be Suppressed in Expanded Vδ1 T Cells

Clinical data shows that Vδ1 T cells are a strong positive prognostic, and therefore an attractive target for use in cell therapy. However, our data show that Vδ1 can be pro- or anti-tumor and using a published expansion protocol gives rise to a proportion of cells with a wound healing phenotype, which may be counterproductive for their in vivo efficacy against cancer. Therefore, we aimed to design a technique to expand cytotoxic Vδ1 cells and eliminate AREG producing cells (FIG. 6A). We found that IL-15 significantly reduced AREG production and increased IFNγ production (FIGS. 6A and 6B). Next, we designed an optimal Vδ1 expansion protocol to enhance proliferation, suppress AREG, and limit cell exhaustion. We found that the combination of IL-15, IL-18, anti-CD3 and anti-CD2 provided the optimal balance of cell expansion and promotion of an anti-tumor phenotype, which we term 2^{nd} generation (Gen 2). This new combination enhanced expansion of Vδ1 cells compared to Gen 1 (FIG. 6A). RNA-seq analysis of activated Gen 1 and Gen 2 Vδ1 cells, showed an enrichment of genes associated with NK cell receptor mediated cytotoxicity, cytokine production, antigen processing/presentation and lysosome formation. We confirmed that Gen 2 cells upregulated more NK cell receptors compared to Gen 1 cells, including CD16, NKG2D and NKp44, but NKp80 expression was reduced in Gen 2 cells (FIG. 6D). Most Gen 1 cells expressed markers of naïve or central memory, while Gen 2 cells were enriched for effector memory and TEMRA markers (FIGS. 6E-6F). Expansion through cell activation can often lead to the accumulation of exhausted effector cells, which are unsuitable for immunotherapy. Gen 2 cells have increased TIGIT expression (FIG. 6G, which was associated with improved function in tumor infiltrating Vδ1 in both CRC tumor datasets (FIGS. 5D-5E). TIM3 and LAG3 were decreased in Gen 2 cells (FIGS. 6H-6I). PD-1 was expressed at low levels in both expansion protocols (FIG. 6J), highlighting again that PD1 is not typically associated with human Vδ1 T cells. Functionally, Vδ1 from the Gen 2 protocol had enhanced cytotoxicity against leukemia cell lines (FIG. 6K). In addition, Gen 2 cells displayed higher levels of cytotoxicity against colorectal cancer cells (SW480) and increased levels of CD107a, a marker of degranulation (FIGS. 6K and 6P). Furthermore, Gen 2 cells produce more IFNγ and less AREG compared to Gen 1 cells (FIGS. 6L-6N). Using the scratch wound healing assay, we next investigated the ability of Gen 2 cells to prevent tumor cell migration and prevent the ‘wound’ closing. After 6 hours, Gen 2 cells prevented tumor cell growth compared to Gen 1 cells. At 24 hours, Gen 2 cells further prevented wound healing and tumor growth. Indeed, in 5 out of 8 donors, Gen 2 cells increased the wound area, while Gen 1 cells reduced the wound area in both SW480 and HCT160 cells (FIG. 6O). We hypothesized that this was due to direct killing of tumor cells by Gen 2 Vδ1 cells. During the assay, Gen 2 cells expressed significantly more CD107a, a marker of degranulation (FIG. 6P), and higher levels of IFNγ compared to Gen 1 (FIG. 6Q). Lastly, Gen 2 cells produced significantly less AREG in response to scratched tumor cells (FIG. 6R). In summary, Vδ1 T cells produced with the Gen 2 protocol have increased proliferation, reduced expression of exhaustion markers, increased cytotoxic function and do not contribute to wound repair, as seen in Gen 1 cells.

### Example 8. Enhanced Function of Gen 2 Vδ1 Cells is Underpinned by Improved Metabolic Activity

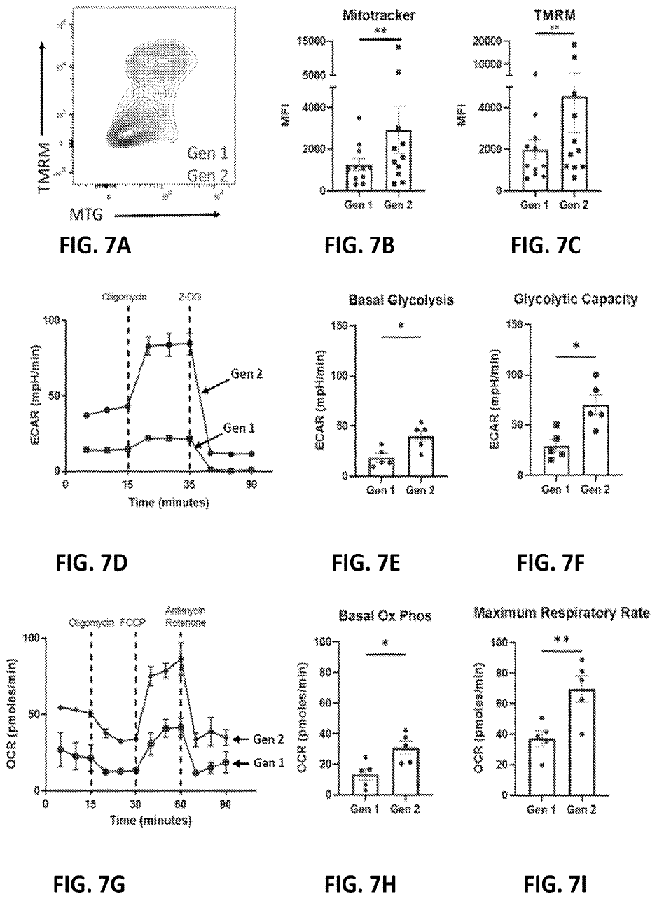

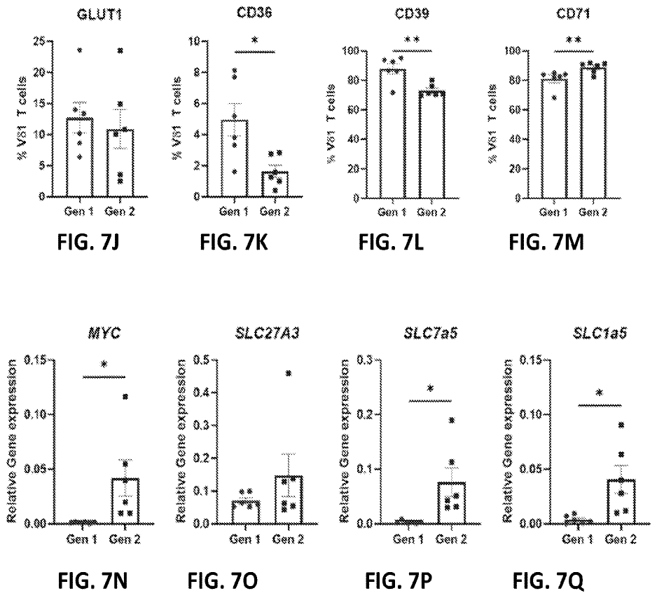

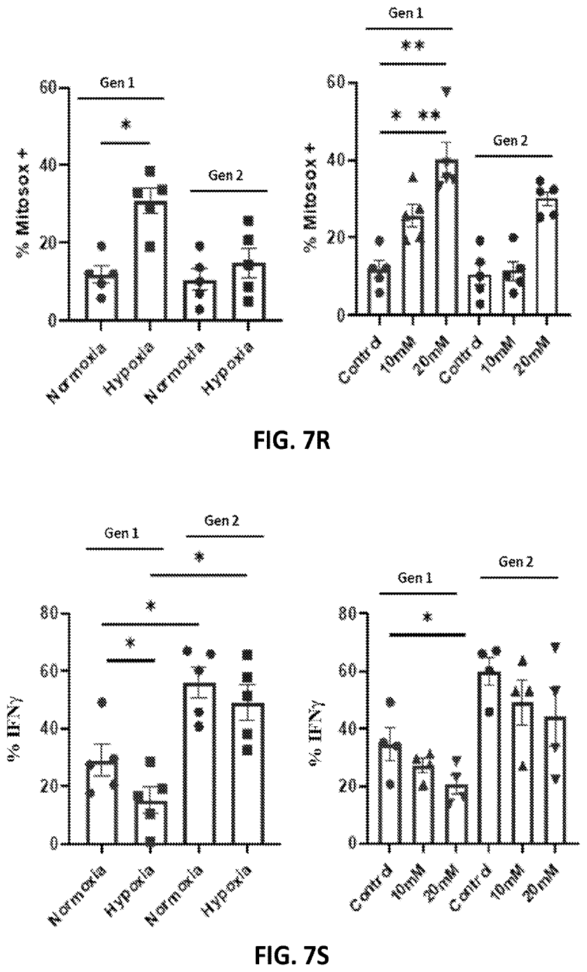

Immune cell function is intricately linked with cellular metabolism. Extensive data now shows that immune cells ramp up their metabolic activity to proliferate, and to fuel effector functions (Poznanski et al., Cell Metab. 33, 1205-1220.e5 (2021); Lunt et al., Annu. Rev. Cell Dev. Biol. 27, 441-64 (2011)). Furthermore, different metabolic programs are associated with cytotoxicity, longevity, and cytokine production (Caraman et al., Immunity 48, 812-830.e14 (2018)). We analyzed if there were cellular metabolic differences between Gen 1 and Gen 2 cells. Firstly, Gen 2 cells showed increased mitochondrial mass and membrane potential (FIGS. 7A-7C). Metabolic flux analysis of Vδ1 cells demonstrated that Gen 2 cells maintained higher levels of basal glycolysis and had a significantly higher glycolytic capacity compared to Gen 1 cells (FIGS. 7D-7F), as well as higher basal oxidative phosphorylation and maximum respiratory rate (FIGS. 7G-7I). Nutrient transporters were analyzed by flow cytometry, including GLUT1, CD36, CD39 and CD71 (FIGS. 7J-7M). Gen 1 cells expressed higher levels of CD36, associated with lipid uptake which is linked to ferroptosis in the TME (Xu et al., Immunity 1-17 (2021) doi:10.1016/j.immuni.2021.05.003; Ma et al., Cell Metab. 1-12 (2021) doi:10.1016/j.cmet.2021.02.015)). Gen 2 cells had slightly but significantly higher expression of iron transporter CD71, essential for T cell function. At the transcriptional level, MYC, a key metabolic master regulator was significantly higher in Gen 2 cells (FIG. 7N), as well as the amino acid transporter SLC7a5 and SLC7A1 and glutamine transporter SLC1A5 (FIGS. 7O-Q). Collectively these results show that Gen 2 cells are more metabolically fit than Gen 1 cells, and metabolic fitness has been shown to be important for anti-tumor functions in TME which is often not replete in nutrients and oxygen (Combes et al., Cell 185, 184-203.e19 (2022); Baginska et al., Front. Immunol. 4, 490 (2013)). In order to test this, we cultured Gen 1 and Gen 2 cells in increasing concentrations of lactic acid (0-20 mM) or in a hypoxic environment (0.5% O2). While Gen1 cells increased production of mitochondrial ROS (mitosox) in acidic and hypoxic environments, Gen 2 cells did not significantly increase their ROS production (FIG. 7R). Functionally, this allowed Gen 2 cells to maintain IFNγ production in these hostile environments, while Gen 1 cells decreased effector function (FIG. 7S).

### Example 9. GEN 2 Cells Control PDX and Persist for Longer Due to Maintenance of Metabolic Fitness

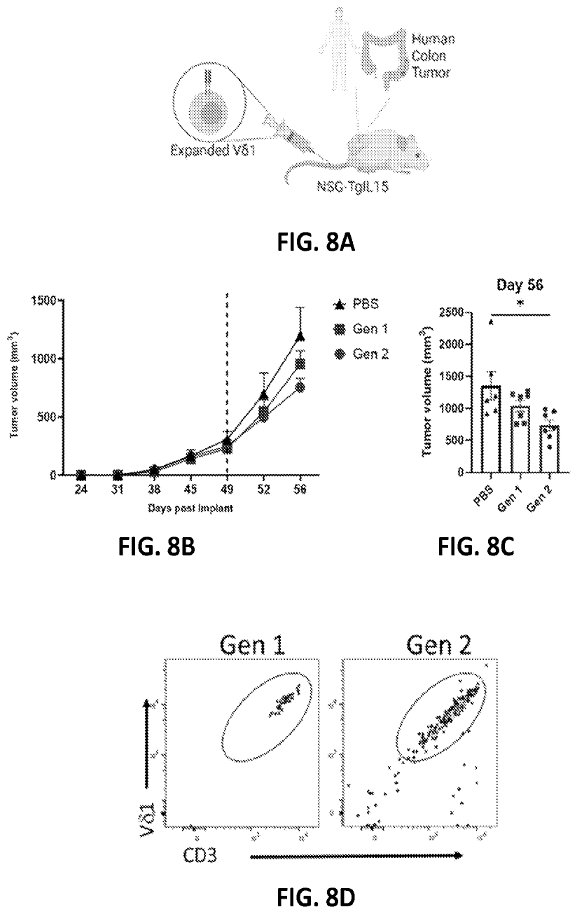

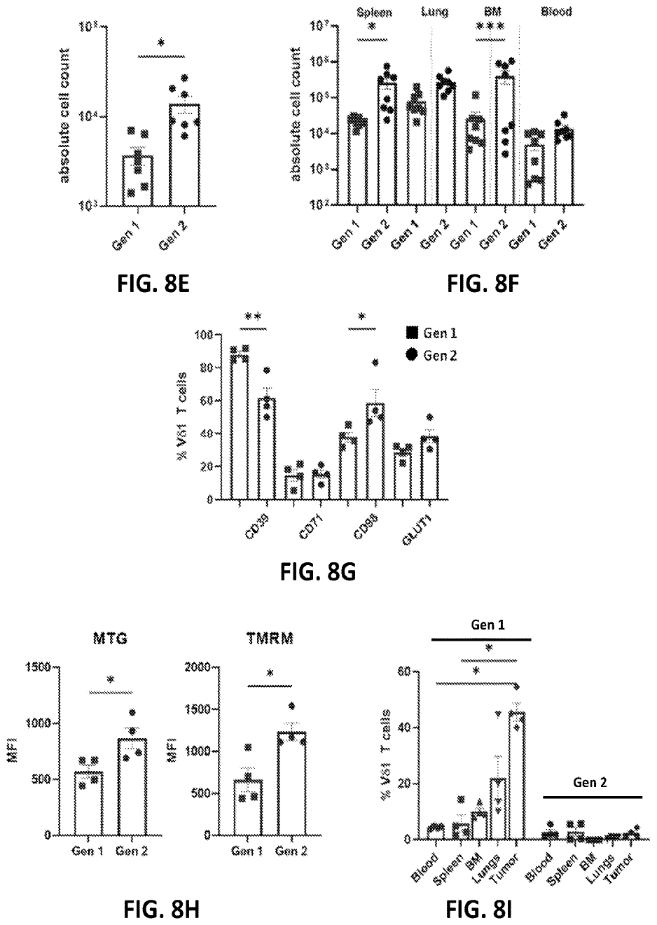

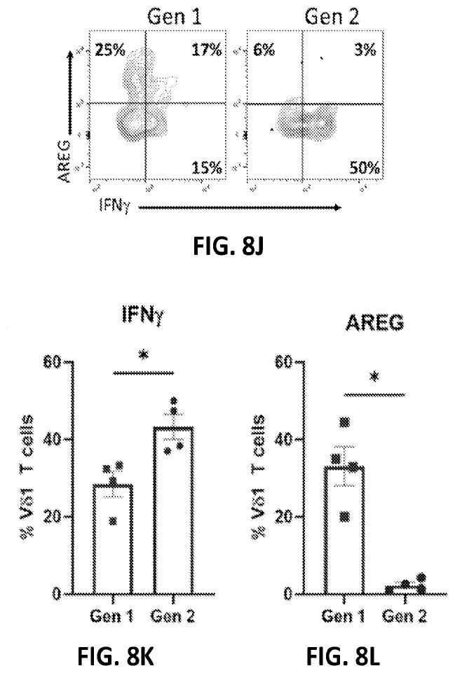

Gen 2 outperform Gen 1 cells for cytotoxicity and metabolic fitness in vitro. To investigate the efficacy of Vδ1 cells against tumors in vivo we employed a patient derived xenograft model of CRC (FIG. 8A). Human CRC tumors were implanted in NSG-Tg(HuIL15) mice. After 6 weeks, when tumor growth was established, 10×10⁶ Vδ1 cells, expanded using either Gen 1 or Gen 2 protocols were transferred into tumor bearing mice. Gen 2 cells significantly reduced tumor growth compared to PBS controls (FIGS. 8B-8C). Gen 1 cells showed less efficacy, as they contain a mixed population of cytotoxic and AREG producers. Importantly, Gen 2 cells were found at significantly higher numbers in tumors compared to Gen 1 cells. Indeed there were log-fold or greater increase in Gen 2 cells in the tumor, as well as peripheral organs including spleen and bone marrow (FIGS. 8D-8F). This may be due to their increased expression of trafficking and tissue residency markers, such as CD69, CXCR3, CXCR6 and CCR5. Gen 2 cells maintained their enhanced metabolic profile seen during expansion, including reduced CD39 expression and increased expression of CD98 (FIG. 8G). Furthermore, Gen 2 cells maintained improved mitochondrial dynamics in the TME, with higher mitochondrial mass and membrane potential (FIGS. 8H-8J). Remarkably, Gen 2 cells did not produce AREG in any organs, including the tumor. In contrast, the TME strongly induced the production of AREG from Gen 1 cells, as did the lung (FIG. 8J). Furthermore, tumor infiltrating Gen 2 cells produced significantly more IFNγ, but AREG was absent from Gen 2 cells but highly expressed by Gen 1 cells (FIGS. 8K-8M). In summary, we developed an expansion protocol to eliminate the expansion of AREG producing Vδ1, which have enhanced metabolic fitness, and this phenotype is maintained in vivo in the harsh tumor environment, which induces AREG from other cell types.

### Example 10. Identification of Nutrients that Affect AREG Producing Vδ1 Cells

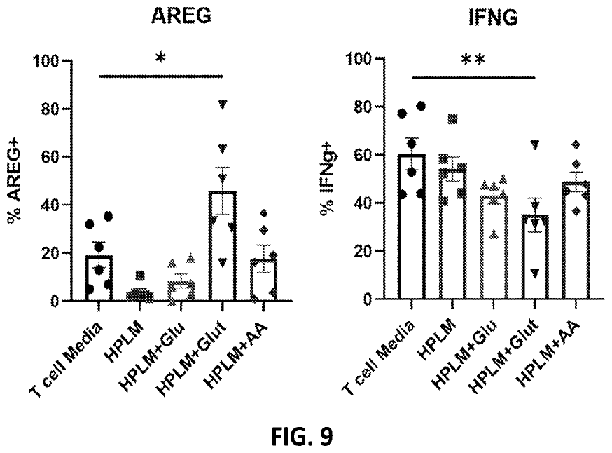

To identify nutrients that can affect AREG production, Vδ1 cells were expanded using Gen 1 protocol. The cells were then transferred to human plasma-like media (HPLM) that has low levels of nutrients. To check the effect of different nutrients on AREG production, the cells were cultured in HPLM alone, HPLM supplemented with excess glucose (Glu 1 mM; “HPLM+Glu”), HPLM supplemented with glutamine (Glut, 2 mM; “HPLM+Glut”), or HPLM supplemented with non-essential MEM amino acids (AA, 1:50 dilution; “HPLM+AA”). The amino acids included L-Arginine hydrochloride 29.952606 mM; L-Cystine 5 mM; L-Histidine hydrochloride-H2O 10 mM; L-Isoleucine 20 mM; L-Leucine 20 mM; L-Lysine hydrochloride 19.808743 mM; L-Methionine 5.067114 mM; L-Phenylalanine 10 mM; L-Threonine 20 mM; L-Tryptophan 2.5 mM; L-Tyrosine 9.944752 mM; and L-Valine 20 mM. The cells were then studied for AREG and IFNg production. The results are shown in FIG. 9.

As described in FIG. 9, AREG production was significantly promoted in Vδ1 cells that were cultured in medium supplemented with glutamine (HPLM+Glut). On the other hand, IFNg production was markedly reduced in these cells. Thus presence of additional glutamine in culture medium promoted AREG producing Vδ1 cells (that have wound healing phenotype) and inhibited IFNg producing Vδ1 cells.

Accordingly, the present Example demonstrates glutamine as a nutrient that can promote AREG producing Vδ1 cells and inhibit IFNg producing Vδ1 cells.

### REFERENCES

1. Siegel, R. L. et al. Colorectal cancer statistics, 2020. CA. Cancer J. Clin. 70, 145-164 (2020).
2. Guinney, J. et al. The consensus molecular subtypes of colorectal cancer. Nat. Med. 21, 1350-6 (2015).
3. Galon, J. et al. Towards the introduction of the ‘Immunoscore’ in the classification of malignant tumours. J. Pathol. 232, 199-209 (2014).
4. Li, X. et al. The immunological and metabolic landscape in primary and metastatic liver cancer. Nat. Rev. Cancer 21, 541-557 (2021).
5. Fontana, E., Eason, K., Cervantes, A., Salazar, R. & Sadanandam, A. Context matters-consensus molecular subtypes of colorectal cancer as biomarkers for clinical trials. Ann. Oncol. 30, 520-527 (2019).
6. Thorsson, V. et al. The Immune Landscape of Cancer. Immunity 48, 812-830.e14 (2018).
7. Singh, P. P., Sharma, P. K., Krishnan, G. & Lockhart, A. C. Immune checkpoints and immunotherapy for colorectal cancer. Gastroenterol. Rep. 3, gov053 (2015).
8. Nielsen, M. M., Witherden, D. A. & Havran, W. L. γδ T cells in homeostasis and host defence of epithelial barrier tissues. Nat. Rev. Immunol. 17, 733-745 (2017).
9. Melsen, J. E., Lugthart, G., Lankester, A. C. & Schilham, M. W. Human Circulating and Tissue-Resident CD56bright Natural Killer Cell Populations. Front. Immunol. 7, 1-10 (2016).
10. Schenkel, J. M. & Masopust, D. Tissue-Resident Memory T Cells. Immunity 41, 886-897 (2014).
11. Vivier, E. et al. Innate Lymphoid Cells: 10 Years On. Cell 174, 1054-1066 (2018).
12. Suzuki, T., Hayman, L., Kilbey, A., Edwards, J. & Coffelt, S. B. Gut γδ T cells as guardians, disruptors, and instigators of cancer. Immunol. Rev. 298, 198-217 (2020).
13. Raverdeau, M., Cunningham, S. P., Harmon, C. & Lynch, L. γδ T cells in cancer: a small population of lymphocytes with big implications. Clin. Transl. Immunol. 8, (2019).
14. Gentles, A. J. et al. The prognostic landscape of genes and infiltrating immune cells across human cancers. Nat. Med. 21, 938-945 (2015).
15. Wu, D., Wu, P., Qiu, F., Wei, Q. & Huang, J. Human γδT-cell subsets and their involvement in tumor immunity. Cell. Mol. Immunol. 14, 245-253 (2017).
16. Wu, P. et al. γδT17 Cells Promote the Accumulation and Expansion of Myeloid-Derived Suppressor Cells in Human Colorectal Cancer. Immunity 40, 785-800 (2014).
17. Holderness, J., Hedges, J. F., Ramstead, A. & Jutila, M. A. Comparative Biology of γδ T Cell Function in Humans, Mice, and Domestic Animals. Annu. Rev. Anim. Biosci. 1, 99-124 (2013).
18. Raverdeau, M., Cunningham, S. P., Harmon, C. & Lynch, L. γδ T cells in cancer: a small population of lymphocytes with big implications. Clin. Transl. Immunol. 8, 1-15 (2019).
19. Hoeres, T., Smetak, M., Pretscher, D. & Wilhelm, M. Improving the efficiency of Vγ9Vδ2 T-cell immunotherapy in cancer. Front. Immunol. 9, 1-18 (2018).
20. Meraviglia, S. et al. Distinctive features of tumor-infiltrating γδ T lymphocytes in human colorectal cancer. Oncoimmunology 6, (2017).
21. Flier, J. S., Underhill, L. H. & Dvorak, H. F. Tumors: Wounds That Do Not Heal. N. Engl. J. Med. 315, 1650-1659 (1986).
22. Dvorak, H. F. Tumors: wounds that do not heal-redux. Cancer Immunol. Res. 3, 1-11 (2015).
23. Pelka, K. et al. Spatially organized multicellular immune hubs in human colorectal cancer. Cell 184, 4734-4752.e20 (2021).
24. Chan, C. J. et al. The receptors CD96 and CD226 oppose each other in the regulation of natural killer cell functions. Nat. Immunol. (2014).
25. Llosa, N. J. et al. The vigorous immune microenvironment of microsatellite instable colon cancer is balanced by multiple counter-inhibitory checkpoints. Cancer Discov. 5, 43-51 (2015).
26. Boland, R. C. & Goel, A. Microsatellite Instability in Colorectal Cancer. Gastroenterology 138, 2073-2087 (2010).
27. Anitei, M.-G. et al. Prognostic and predictive values of the immunoscore in patients with rectal cancer. Clin. Cancer Res. 20, 1891-9 (2014).
28. Almeida, A. R. et al. Delta one T cells for immunotherapy of chronic lymphocytic leukemia: Clinical-grade expansion/differentiation and preclinical proof of concept. Clin. Cancer Res. 22, 5795-5804 (2016).
29. Poznanski, S. M. et al. Metabolic flexibility determines human NK cell functional fate in the tumor microenvironment. Cell Metab. 33, 1205-1220.e5 (2021).
30. Lunt, S. Y. & Vander Heiden, M. G. Aerobic glycolysis: meeting the metabolic requirements of cell proliferation. Annu. Rev. Cell Dev. Biol. 27, 441-64 (2011).
31. Caraman, I. et al. The Immune Landscape of Cancer. Immunity 48, 812-830.e14 (2018).
32. Xu, S. et al. Uptake of oxidized lipids by the scavenger receptor CD36 promotes lipid peroxidation and dysfunction in CD8+ T cells in tumors. Immunity 1-17 (2021) doi:10.1016/j.immuni.2021.05.003.
33. Ma, X. et al. CD36-mediated ferroptosis dampens intratumoral CD8+ T cell effector function and impairs their antitumor ability. Cell Metab. 1-12 (2021) doi:10.1016/j.cmet.2021.02.015.
34. Combes, A. J. et al. Discovering dominant tumor immune archetypes in a pan-cancer census. Cell 185, 184-203.e19 (2022).
35. Baginska, J. et al. The Critical Role of the Tumor Microenvironment in Shaping Natural Killer Cell-Mediated Anti-Tumor Immunity. Front. Immunol. 4, 490 (2013).
36. Bridgewater, J. A. et al. Systemic chemotherapy with or without cetuximab in patients with resectable colorectal liver metastasis (New EPOC): long-term results of a multicentre, randomised, controlled, phase 3 trial. Lancet Oncol. 21, 398-411 (2020).
37. Jonker, D. J. et al. Cetuximab for the Treatment of Colorectal Cancer. N. Engl. J. Med. 357, 2040-2048 (2007).
38. Larson, R. C. et al. CAR T cell killing requires the IFNγR pathway in solid but not liquid tumours. Nature (2022) doi:10.1038/s41586-022-04585-5.
39. Mayassi, T. et al. Chronic Inflammation Permanently Reshapes Tissue-Resident Immunity in Celiac Disease. Cell 176, 967-981.e19 (2019).
40. Guo, X.-Z. J. et al. Lung γδ T Cells Mediate Protective Responses during Neonatal Influenza Infection that Are Associated with Type 2 Immunity. Immunity 49, 531-544.e6 (2018).
41. Agaronyan, K. et al. Tissue remodeling by an opportunistic pathogen triggers allergic inflammation. Immunity 1-17 (2022) doi:10.1016/j.immuni.2022.04.001.
42. Jin, C. et al. Commensal Microbiota Promote Lung Cancer Development via γδ T Cells. Cell 176, 998-1013.e16 (2019).
43. Yu, X. et al. The surface protein TIGIT suppresses T cell activation by promoting the generation of mature immunoregulatory dendritic cells. Nat. Immunol. 10, 48-57 (2009).
44. Marin-Acevedo, J. A. et al. Next generation of immune checkpoint therapy in cancer: new developments and challenges. J. Hematol. Oncol. 11, 39 (2018).
45. Ge, Z., Peppelenbosch, M. P., Sprengers, D. & Kwekkeboom, J. TIGIT, the Next Step Towards Successful Combination Immune Checkpoint Therapy in Cancer. Front. Immunol. 12, (2021).
46. Harjunpaa, H. & Guillerey, C. TIGIT as an emerging immune checkpoint. Clin. Exp. Immunol. 200, 108-119 (2020).
47. Pizzolato, G. et al. Single-cell RNA sequencing unveils the shared and the distinct cytotoxic hallmarks of human TCRVδ1 and TCRVδ2 γδ T lymphocytes. Proc. Natl. Acad. Sci. 2019, 201818488 (2019).
48. Freud, A. G. et al. NKp80 Defines a Critical Step during Human Natural Killer Cell Development. Cell Rep. 16, 379-391 (2016).
49. Vitale, M. et al. Identification of NKp80, a novel triggering molecule expressed by human NK cells. Eur. J. Immunol. 31, 233-42 (2001).
50. Kuttruff, S. et al. NKp80 defines and stimulates a reactive subset of CD8 T cells. Blood 113, 358-69 (2009).
51. Di Lorenzo, B. et al. Broad Cytotoxic Targeting of Acute Myeloid Leukemia by Polyclonal Delta One T Cells. Cancer Immunol. Res. 7, 552-558 (2019).
52. Lenz, H.-J. et al. Impact of Consensus Molecular Subtype on Survival in Patients With Metastatic Colorectal Cancer: Results From CALGB/SWOG 80405 (Alliance). J. Clin. Oncol. 37, 1876-1885 (2019).
53. Terrén, I. et al. Metabolic changes of Interleukin-12/15/18-stimulated human NK cells. Sci. Rep. 11, 6472 (2021).
54. Cooper, M. A. et al. Cytokine-induced memory-like natural killer cells. Proc. Natl. Acad. Sci. 106, 1915-1919 (2009).
55. Zhou, T. et al. IL-18BP is a secreted immune checkpoint and barrier to IL-18 immunotherapy. Nature 583, 609-614 (2020).
56. Scharping, N. E. et al. The Tumor Microenvironment Represses T Cell Mitochondrial Biogenesis to Drive Intratumoral T Cell Metabolic Insufficiency and Dysfunction. Immunity 45, 374-388 (2016).
57. Yu, Y. R. et al. Disturbed mitochondrial dynamics in CD8+ TILs reinforce T cell exhaustion. Nat. Immunol. (2020) doi:10.1038/s41590-020-0793-3.
58. Qiu, J. et al. Acetate Promotes T Cell Effector Function during Glucose Restriction. Cell Rep. 27, 2063-2074.e5 (2019).
59. Buck, M. D., O'Sullivan, D. & Pearce, E. L. T cell metabolism drives immunity. J. Exp. Med. 212, 1345-1360 (2015).
60. Michelet, X. et al. Metabolic reprogramming of natural killer cells in obesity limits antitumor responses. Nat. Immunol. 19, 1330-1340 (2018).
61. Krishnan, S. et al. Amphiregulin-producing γδ T cells are vital for safeguarding oral barrier immune homeostasis. Proc. Natl. Acad. Sci. 115, 10738-10743 (2018).
62. Guo, X. zhi J. et al. Lung γδ T Cells Mediate Protective Responses during Neonatal Influenza Infection that Are Associated with Type 2 Immunity. Immunity 49, 531-544.e6 (2018).
63. Zaiss, D. M. W., Gause, W. C., Osborne, L. C. & Artis, D. Emerging functions of amphiregulin in orchestrating immunity, inflammation, and tissue repair. Immunity 42, 216-226 (2015).
64. Liu, J. et al. An Integrated TCGA Pan-Cancer Clinical Data Resource to Drive High-Quality Survival Outcome Analytics. Cell 173, 400-416.e11 (2018).
65. Maykel, J. et al. NOD-scidIl2rg (tm1Wjl) and NOD-Rag1 (null) Il2rg (tm1Wjl): a model for stromal cell-tumor cell interaction for human colon cancer. Dig. Dis. Sci. 59, 1169-79 (2014).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for expansion of a population of γδ T cells of Vδ1 subtype (Vδ1 T cells), the method comprising:
(i) providing a population of cells comprising Vδ1 T cells isolated from a human subject;
(ii) culturing the Vδ1 T cells from step (i) in a first medium comprising human serum, interleukin 1β (IL-1β), IL-4, IL-21, interferon gamma (IFNγ), and an anti-CD3 antibody or an antigen-binding fragment thereof, for 10-20 days, optionally wherein the first medium further comprises glutamine; and
(iii) culturing the cells in a second medium comprising human serum, IL-15, IFNγ, IL18, an anti-CD2 antibody or an antigen-binding fragment thereof, and an anti-CD3 antibody or an antigen-binding fragment thereof, for 3-10 days, optionally wherein the second medium further comprises glutamine.

2. The method of claim 1, wherein the population of Vδ1 T cells are isolated from a biological sample from the human subject.

3. The method of claim 2, wherein isolating the population of Vδ1 T cells from the biological sample comprises isolation of a population of peripheral blood mononuclear cells (PBMCs) from the biological sample and depletion of monocytes, macrophages, and/or alpha beta T cells from the population of PBMCs.

4. The method of claim 3, wherein isolation of the population of PBMCs from the biological sample comprises density gradient centrifugation.

5. The method of claim 2, wherein the biological sample comprises blood.

6. The method of claim 1, further comprising identification of the Vδ1 T cells by immunophenotyping.

7. The method of claim 6, wherein the identification of the Vδ1 T cells comprises: detecting the presence of one or more of TCR Vδ1, CD3, NKG2D, γδTCR, and TIGIT on the Vδ1 T cells; and/or detecting the absence of one or more of CD14, CD68, CD19, CD21, CD56, and CD4 on the Vδ1 T cells.

8. (canceled)

9. The method of claim 6, wherein the immunophenotyping comprises flow cytometry or immunofluorescence microscopy.

10. The method of claim 1, wherein the cells are cultured in the first medium for 14 or 15 days, and/or in the second medium for 7 or 8 days.

11. (canceled)

12. The method of claim 1, wherein the first medium comprises about 1-10% human serum, about 10-20 ng/ml IL-1β, about 50-150 ng/ml IL-4, about 1-15 ng/ml IL-21, about 10-150 ng/ml IFNγ, and about 10-150 ng/ml of the anti-CD3 antibody or antigen-binding fragment thereof.

13. (canceled)

14. The method of claim 1, wherein the first medium further comprises glutamine.

15. The method of claim 14, wherein the first medium comprises about 1-5 mmol glutamine, optionally wherein the first medium comprises about 2 mmol glutamine.

16. (canceled)

17. The method of claim 1, wherein the second medium comprises about 0.1%-5% human serum, about 10-150 ng/ml IL-15, about 1-50 ng/ml IFNγ, about 1-100 ng/ml IL-18, about 100-1000 ng/ml of the anti-CD2 antibody or antigen-binding fragment thereof, and about 0.1-10 μg/ml of the anti-CD3 antibody or antigen-binding fragment thereof.

18. (canceled)

19. The method of claim 1, wherein the second medium further comprises glutamine.

20. The method of claim 19, wherein the second medium comprises about 1-5 mmol glutamine, optionally wherein the second medium comprises about 2 mmol glutamine.

21. (canceled)

22. An expanded population of Vδ1 T cells obtained by the method of claim 1.

23. The expanded population of Vδ1 T cells of claim 22, wherein 35% or more cells of the population express T cell immunoreceptors with Ig and ITIM domains (TIGIT)_{z}35% or more cells of the population express IFNγ, 20% or less cells of the population express NKp80, 20% or less cells of the population express AREG, and/or 35% or more cells of the population express cytotoxic effector molecules when co-incubated with cancer cells.

24.-27. (canceled)

28. The expanded population of Vδ1 T cells of claim 23, wherein the cytotoxic effector molecules comprise one or more of granzyme B, perforin, IFNγ, CD107a, TNFα, RANTES, granzyme K, TRAIL, FAS-FASL, and granulysin.

29.-30. (canceled)

31. A method of treating cancer in a subject in need thereof, the method comprising administering to the subject a therapeutically effective amount of the expanded population of Vδ1 T cells of claim 22.

32. The method of claim 31, wherein the population of Vδ1 T cells is a population of allogeneic Vδ1 T cells or a population of autologous Vδ1 T cells.

33. (canceled)

34. The method of claim 31, wherein the cancer is a solid tumor.

35. The method of claim 31, wherein the subject is a human.
